(19) United States
(12) Patent Application Publication
(10) Pub. No.: US 2025/0186349 A1
(43) Pub. Date: Jun. 12, 2025

(54) LYOPHILIZED FORMULATION, AND PREPARATION METHOD THEREFOR AND USE THEREOF

(71) Applicant: ZHUHAI BEIHAI BIOTECH CO., LTD., Zhuhai, Guangdong (CN)

(72) Inventors: Qun Sun, Zhuhai, Guangdong (CN); Bobo Hu, Zhuhai, Guangdong (CN)

(21) Appl. No.: 18/844,056

(22) PCT Filed: Mar. 15, 2023
(86) PCT No.: PCT/CN2023/081625

§ 371 Date: Sep. 5, 2024

(30) Foreign Application Priority Data
Mar. 17, 2022 (CN) 202210265978.5

(51) Int. Cl. A61K 9/19; A61K 31/675; A61K 38/38

(52) U.S. Cl. CPC A61K 9/19; A61K 31/675; A61K 38/385

(57) ABSTRACT

The present invention relates to the field of pharmaceutical technology, and particularly provided are a lyophilized formulation, and a preparation method therefor and the use thereof. The lyophilized formulation comprises fosaprepitant or a pharmaceutically acceptable salt thereof and human serum albumin, wherein the weight ratio of fosaprepitant or a pharmaceutically acceptable salt thereof to human serum albumin is 1:0.2 to 1:2. The lyophilized formulation may further comprise palonosetron or a pharmaceutically acceptable salt thereof, a lyophilization excipient and a pH adjuster.

### TECHNICAL FIELD

The present invention relates to the field of pharmaceutical technology, specifically addressing a lyophilized formulation for treating and preventing nausea and vomiting caused by tumor chemotherapy, and more particularly relates to a lyophilized formulation comprising fosaprepitant or a pharmaceutically acceptable salts thereof and, if necessary, also including palonosetron or a pharmaceutically acceptable salt thereof.

### BACKGROUND

Tumor is a disease that poses serious threat to human life and quality of life, and chemotherapy is a primary treatment modality for treating malignant tumors. The most common side effect of chemotherapy is nausea and vomiting (CINV), which is one of the most painful adverse effects widely believed by tumor patients. Poor control of CINV can affect patient's functional status and quality of life. Patient may delay scheduled chemotherapy or even sometimes reject potentially therapeutically effective treatments due to CINV.

Fosaprepitant is a selective high-affinity blocker against substance P/neurokinin 1 (NK-1) receptors and functions primarily by blocking cerebral nausea and vomiting signals. Palonosetron is a selective 5-hydroxytryptamine 3 receptor (5-HT3) antagonist and has high selective antagonistic action on 5-HT3 receptor. It can block the excitation of presynaptic 5-HT3 receptor of central and peripheral neurons of vomiting reflex, directly influence the action of the vagus nerve in transmitting 5-HT3 receptor to the area postrema in the central nervous system, block the vagus nerve terminals in the intestinal tract, prevent signal transmission to the trigger zone of 5-HT3 receptor, and reduce the occurrence rate of vomiting and nausea, but has poor effect on nausea and vomiting that have appeared. Fosaprepitant is usually used in combination with palonosetron clinically to prevent and treat acute and delayed nausea and vomiting caused by moderately emetic and severely emetic anticancer drugs at the initial phase of chemotherapy or after repeated administration.

Fosaprepitant is a water-soluble phosphoryl prodrug of aprepitant. Following intravenous administration, it is rapidly converted to aprepitant in vivo by phosphatases ubiquitously in the body. The antiemetic effect of fosaprepitant is attributed to aprepitant. Unless stored at a low temperature, fosaprepitant is easily degraded into aprepitant, and the degradation effect is more obvious in an aqueous solution. However, aprepitant is insoluble in water. To dissolve aprepitant which may exist, a solubilizer needs to be added into a formulation to ensure that the formulation does not precipitate particles for a long enough time after being formulated into a drug solution for clinical use, so that injection administration can be completed.

Polysorbate 80 (Tween 80) is added as a solubilizer in the current fosaprepitant formulation formulas. Polysorbate 80 can change the fluidity of cell membranes, causing increased membrane permeability and thereby leading to severe allergic reactions and cumulative fluid retention; in addition, polysorbate 80 itself may also cause hemolytic reactions and cholestasis.

In view of the above technical drawbacks, the patent “FORMULATIONS OF FOSAPREPITANT AND APREPITANT” (patent No. CN 109789154 B) discloses a fosaprepitant and aprepitant composition/formulation that is free of polysorbate 80. Instead, it selects human serum albumin as a safer solubilizer, solving the problem of toxic and side effects caused by polysorbate 80, but does not consider the problem caused by the addition amount of human serum albumin.

Further research in the present invention finds that along with the increase of the addition amount of human serum albumin, the reconstitution time of the lyophilized formulation becomes longer, a large number of bubbles which are difficult to dissipate are prone to be generated, and even the risk of incomplete reconstitution exists. Additionally, the storage stability of the finished product becomes worse, and the growth rate of aprepitant is significantly accelerated along with the increase of human serum albumin, so that the content of aprepitant in the finished product is too high, and the risk of precipitating particles after reconstitution is increased, thus resulting in corresponding clinical safety problems.

The long reconstitution time of the product causes inconvenience for busy medical personnel in administering the drug promptly to patients. A large number of generated bubbles are not dissipated after a long time (no less than 2 h) makes it challenging to draw the solution with a syringe, leading to a reduced volume of drug solution available for use and consequently a lower drug concentration after dilution. Incomplete reconstitution and poor storage stability of the finished product further raise concerns regarding drug safety and efficacy.

To solve the above problems caused by excessive human serum albumin in the lyophilized formulations of the present invention, a large number of experiments were conducted. Various proportions of human serum albumin and numerous injection excipients were tested to develop the technical solution presented in the present invention.

### Technical Problem

Aiming at the defects of the prior art, the present invention provides a lyophilized formulation (composition) containing fosaprepitant or a pharmaceutically acceptable salt thereof and human serum albumin, along with methods for its preparation and clinical use. The lyophilized formulation is characterized by a short reconstitution time. good storage stability, proven safety and efficacy. Additionally, it features a simple preparation process that is suitable for industrial-scale production. The lyophilized formulation solves a series of problems including long reconstitution time, generation of a large number of bubbles during reconstitution, inability to guarantee complete reconstitution within a certain time, poor storage stability of finished products, etc.

### Technical solution

To obtain a safe and effective lyophilized formulation with a short reconstitution time and good storage stability, the present invention involved extensive innovative testing of the amounts of human serum albumin, lyophilization excipients, and pH adjuster, and it is unexpectedly found that:
(1) When the weight ratio of fosaprepitant or a pharmaceutically acceptable salt thereof to the human serum albumin is greater than 1:0.2, the human serum albumin cannot achieve an ideal solubilizing effect, a large number of particles are precipitated within 30 min after the product is reconstituted, and potential safety hazards exist if the product is injected into a human body; when the weight ratio of fosaprepitant or the pharmaceutically acceptable salt thereof to the human serum albumin is less than 1:2, a large number of bubbles are generated during reconstitution of the product, a gel lump with very high viscosity is easily formed, the reconstitution time is long, the complete reconstitution within a certain time cannot be guaranteed, and the storage stability of finished products is poor; when the weight ratio of fosaprepitant or the pharmaceutically acceptable salt thereof to the human serum albumin is 1:0.2 to 1:2, the reconstitution time is short, the complete reconstitution within a certain time is guaranteed, the storage stability is good, and an ideal solubilizing effect can be achieved.
(2) A proper amount of the lyophilization excipient can accelerate the reconstitution speed, reduce the degradation of fosaprepitant, and improve the stability of the product during storage.
(3) Adjusting the pH of the drug solution to a suitable range before lyophilization is also conducive to the stability of the product during storage.

Therefore, the present invention provides the following technical solution: a lyophilized formulation comprising fosaprepitant or a pharmaceutically acceptable salt thereof and human serum albumin, wherein the weight ratio of fosaprepitant or the pharmaceutically acceptable salt thereof to the human serum albumin is about 1:0.2 to about 1:2.

In some embodiments, the weight ratio of fosaprepitant or the pharmaceutically acceptable salt thereof to the human serum albumin in the lyophilized formulation is preferably about 1:0.3 to about 1:1.3, such as about 1:0.3 to about 1:1, such as about 1:0.5 to about 1:1.3, such as about 1:0.5 to about 1:1.

In some embodiments, the human serum albumin in the lyophilized formulation is native human serum albumin. In some embodiments, the human serum albumin is recombinant human serum albumin. In some embodiments, the human serum albumin is substantially free of fatty acids. In some embodiments, the human serum albumin is a commercially available human serum albumin solution for infusion.

In some embodiments, the lyophilized formulation further comprises a lyophilization excipient, wherein: the weight ratio of fosaprepitant or the pharmaceutically acceptable salt thereof to the lyophilization excipient is about 1:0.02 to about 1:5; preferably, the weight ratio of fosaprepitant or the pharmaceutically acceptable salt thereof to the lyophilization excipient is about 1:0.2 to about 1:3, such as about 1:0.4 to about 1:2, such as about 1:1 to about 1:3, such as about 1:1 to about 1:2.

In some embodiments, the lyophilized formulation further comprises palonosetron or a pharmaceutically acceptable salt thereof.

In some embodiments, the lyophilized formulation is a lyophilized formulation comprising fosaprepitant or the pharmaceutically acceptable salt thereof and the human serum albumin, and each vial of the lyophilized formulation comprises the following components:
220-270 mg of fosaprepitant dimeglumine;
30-300 mg of the human serum albumin.

In some embodiments, each vial of the lyophilized formulation preferably comprises the following components:
220-270 mg of fosaprepitant dimeglumine;
60-200 mg of the human serum albumin.

In some embodiments, each vial of the lyophilized formulation further preferably comprises the following components:
230-260 mg of fosaprepitant dimeglumine;
100-140 mg of the human serum albumin.

In some embodiments, each vial of the lyophilized formulation comprises the following components:
about 245.3 mg of fosaprepitant dimeglumine;
30-300 mg of the human serum albumin.

In some embodiments, an overfill volume of about 2% to 20% is required in the production process of the lyophilized formulation. In some embodiments, an overfill volume of about 5% to 10% is required in the production process of the lyophilized formulation. In some embodiments, an overfill volume of about 5% is required in the production process of the lyophilized formulation. In some embodiments, an overfill volume of about 10% is required in the production process of the lyophilized formulation. In some embodiments, an overfill volume of about 15% is required in the production process of the lyophilized formulation. In some embodiments, an overfill volume of about 20% is required in the production process of the lyophilized formulation.

In some embodiments, the lyophilized formulation is a lyophilized formulation comprising fosaprepitant or the pharmaceutically acceptable salt thereof, palonosetron or the pharmaceutically acceptable salt thereof, and the human serum albumin, and each vial of the lyophilized formulation comprises the following components:
220-270 mg of fosaprepitant dimeglumine;
0.25-0.31 mg of palonosetron hydrochloride;
30-300 mg of the human serum albumin.

In some embodiments, each vial of the lyophilized formulation preferably comprises the following components:
220-270 mg of fosaprepitant dimeglumine;
0.25-0.31 mg of palonosetron hydrochloride;
60-200 mg of the human serum albumin.

In some embodiments, each vial of the lyophilized formulation further preferably comprises the following components:
230-260 mg of fosaprepitant dimeglumine;
0.26-0.30 mg of palonosetron hydrochloride;
100-140.0 mg of the human serum albumin.

In some embodiments, each vial of the lyophilized formulation comprises the following components:
about 245.3 mg of fosaprepitant dimeglumine;
about 0.28 mg of palonosetron hydrochloride;
30-300 mg of the human serum albumin.

In some embodiments, an overfill volume of about 2% to 20% is required in the production process of the lyophilized formulation. In some embodiments, an overfill volume of about 5% to 10% is required in the production process of the lyophilized formulation. In some embodiments, an overfill volume of about 5% is required in the production process of the lyophilized formulation. In some embodiments, an overfill volume of about 10% is required in the production process of the lyophilized formulation. In some embodiments, an overfill volume of about 15% is required in the production process of the lyophilized formulation. In some embodiments, an overfill volume of about 20% is required in the production process of the lyophilized formulation.

In some embodiments, the lyophilized formulation is a lyophilized formulation comprising fosaprepitant or the pharmaceutically acceptable salt thereof, the human serum albumin, and the lyophilization excipient, and each vial of the lyophilized formulation comprises the following components:
220-270 mg of fosaprepitant dimeglumine;
30-300 mg of the human serum albumin;
15-600 mg of the lyophilization excipient.

In some embodiments, each vial of the lyophilized formulation preferably comprises the following components:
220-270 mg of fosaprepitant dimeglumine;
60-200 mg of the human serum albumin;
60-500 mg of the lyophilization excipient.

In some embodiments, each vial of the lyophilized formulation further preferably comprises the following components:
230-260 mg of fosaprepitant dimeglumine;
100-140 mg of the human serum albumin;
100-400 mg of the lyophilization excipient.

In some embodiments, each vial of the lyophilized formulation comprises the following components:
about 245.3 mg of fosaprepitant dimeglumine;
30-300 mg of the human serum albumin;
10-600 mg of the lyophilization excipient.

In some embodiments, each vial of the lyophilized formulation comprises the following components:
about 245.3 mg of fosaprepitant dimeglumine;
60-200 mg of the human serum albumin;
50-500 mg of the lyophilization excipient.

In some embodiments, an overfill volume of about 2% to 20% is required in the production process of the lyophilized formulation. In some embodiments, an overfill volume of about 5% to 10% is required in the production process of the lyophilized formulation. In some embodiments, an overfill volume of about 5% is required in the production process of the lyophilized formulation. In some embodiments, an overfill volume of about 10% is required in the production process of the lyophilized formulation. In some embodiments, an overfill volume of about 15% is required in the production process of the lyophilized formulation. In some embodiments, an overfill volume of about 20% is required in the production process of the lyophilized formulation.

In some embodiments, the lyophilized formulation is a lyophilized formulation comprising fosaprepitant or the pharmaceutically acceptable salt thereof, palonosetron or the pharmaceutically acceptable salt thereof, the human serum albumin, and the lyophilization excipient. Each vial of the lyophilized formulation comprises the following components:
220-270 mg of fosaprepitant dimeglumine;
0.25-0.31 mg of palonosetron hydrochloride;
30-300 mg of the human serum albumin;
15-600 mg of the lyophilization excipient.

In some embodiments, each vial of the lyophilized formulation preferably comprises the following components:
220-270 mg of fosaprepitant dimeglumine;
0.25-0.31 mg of palonosetron hydrochloride;
60-200 mg of the human serum albumin;
60-500 mg of the lyophilization excipient.

In some embodiments, each vial of the lyophilized formulation further preferably comprises the following components:
230-260 mg of fosaprepitant dimeglumine;
0.26-0.30 mg of palonosetron hydrochloride;
100-140 mg of the human serum albumin;
100-400 mg of the lyophilization excipient.

In some embodiments, each vial of the lyophilized formulation comprises the following components:
about 245.3 mg of fosaprepitant dimeglumine;
about 0.28 mg of palonosetron hydrochloride;

30-300 mg of the human serum albumin;
10-600 mg of the lyophilization excipient.

In some embodiments, each vial of the lyophilized formulation comprises the following components:
about 245.3 mg of fosaprepitant dimeglumine;
about 0.28 mg of palonosetron hydrochloride;
30-300 mg of the human serum albumin;
50-500 mg of the lyophilization excipient.

In some embodiments, an overfill volume of about 2% to 20% is required in the production process of the lyophilized formulation. In some embodiments, an overfill volume of about 5% to 10% is required in the production process of the lyophilized formulation. In some embodiments, an overfill volume of about 5% is required in the production process of the lyophilized formulation. In some embodiments, an overfill volume of about 10% is required in the production process of the lyophilized formulation. In some embodiments, an overfill volume of about 15% is required in the production process of the lyophilized formulation. In some embodiments, an overfill volume of about 20% is required in the production process of the lyophilized formulation.

In some embodiments, the excipient may be all pharmaceutically acceptable excipients.

In some embodiments, the lyophilization excipient is selected from one or more of mannitol, sorbitol, inositol, dextran, maltodextrin, β-cyclodextrin, polyethylene glycol-4000, polyoxyethylene pyrrolidone, sucrose, maltose, lactose, glucose, trehalose, sodium chloride, potassium chloride, calcium chloride, sodium dihydrogen phosphate, hydrolyzed gelatin, glycine, histidine, lysine, alanine, proline, arginine, aspartic acid, asparagine, or sodium glutamate, and is preferably one or two of mannitol and lactose.

In some embodiments, the lyophilized formulation is a lyophilized formulation comprising fosaprepitant or the pharmaceutically acceptable salt thereof, the human serum albumin, and lactose, and the amounts of the components in each vial of the formulation are as follows:
220-270 mg of fosaprepitant dimeglumine;
30-300 mg of the human serum albumin;
10-600 mg of lactose.

In some embodiments, the amounts of the components in each vial of the formulation are preferably as follows:
220-270 mg of fosaprepitant dimeglumine;
60-200 mg of the human serum albumin;
50-500 mg of lactose.

In some embodiments, the amounts of the components in each vial of the formulation are further preferably as follows:
230-260 mg of fosaprepitant dimeglumine;
100-140 mg of the human serum albumin;
100-400 mg of lactose.

In some embodiments, the amounts of the components in each vial of the lyophilized formulation are as follows:
about 245.3 mg of fosaprepitant dimeglumine;
30-300 mg of the human serum albumin;
10-600 mg of lactose.

In some embodiments, the amounts of the components in each vial of the formulation are preferably as follows:
about 245.3 mg of fosaprepitant dimeglumine;
60-200 mg of the human serum albumin;
50-500 mg of lactose.

In some embodiments, an overfill volume of about 2% to 20% is required in the production process of the lyophilized formulation. In some embodiments, an overfill volume of about 5% to 10% is required in the production process of the lyophilized formulation. In some embodiments, an overfill volume of about 5% is required in the production process of the lyophilized formulation. In some embodiments, an overfill volume of about 10% is required in the production process of the lyophilized formulation. In some embodiments, an overfill volume of about 15% is required in the production process of the lyophilized formulation. In some embodiments, an overfill volume of about 20% is required in the production process of the lyophilized formulation.

In some embodiments, the lyophilized formulation is a lyophilized formulation comprising fosaprepitant or the pharmaceutically acceptable salt thereof, the human serum albumin, and mannitol. The amounts of the components in each vial of the formulation are as follows:
220-270 mg of fosaprepitant dimeglumine;
30-300 mg of the human serum albumin;
10-600 mg of mannitol.

In some embodiments, the amounts of the components in each vial of the formulation are preferably as follows:
220-270 mg of fosaprepitant dimeglumine;
60-200 mg of the human serum albumin;
50-500 mg of mannitol.

In some embodiments, the amounts of the components in each vial of the formulation are further preferably as follows:
230-260 mg of fosaprepitant dimeglumine;
100-140 mg of the human serum albumin;
100-400 mg of mannitol.

In some embodiments, the amounts of the components in each vial of the lyophilized formulation are as follows:
about 245.3 mg of fosaprepitant dimeglumine;
30-300 mg of the human serum albumin;
10-600 mg of mannitol.

In some embodiments, an overfill volume of about 2% to 20% is required in the production process of the lyophilized formulation. In some embodiments, an overfill volume of about 5% to 10% is required in the production process of the lyophilized formulation. In some embodiments, an overfill volume of about 5% is required in the production process of the lyophilized formulation. In some embodiments, an overfill volume of about 10% is required in the production process of the lyophilized formulation. In some embodiments, an overfill volume of about 15% is required in the production process of the lyophilized formulation. In some embodiments, an overfill volume of about 20% is required in the production process of the lyophilized formulation.

In some embodiments, the lyophilized formulation is a lyophilized formulation comprising fosaprepitant or the pharmaceutically acceptable salt thereof, palonosetron or the pharmaceutically acceptable salt thereof, the human serum albumin, and lactose. The amounts of the components in each vial of the formulation are as follows:
220-270 mg of fosaprepitant dimeglumine;
0.25-0.31 mg of palonosetron hydrochloride;
30-300 mg of the human serum albumin;
10-600 mg of lactose.

In some embodiments, the amounts of the components in each vial of the formulation are preferably as follows:
220-270 mg of fosaprepitant dimeglumine;
0.25-0.31 mg of palonosetron hydrochloride;
60-200 mg of the human serum albumin;
50-500 mg of lactose.

In some embodiments, the amounts of the components in each vial of the formulation are further preferably as follows:
230-260 mg of fosaprepitant dimeglumine;
0.26-0.30 mg of palonosetron hydrochloride;
100-140 mg of the human serum albumin;
100-400 mg of lactose.

In some embodiments, each vial of the lyophilized formulation comprises the following components:
about 245.3 mg of fosaprepitant dimeglumine;
about 0.28 mg of palonosetron hydrochloride;
30-300 mg of the human serum albumin;
10-600 mg of lactose.

In some embodiments, the amounts of the components in each vial of the formulation are preferably as follows:
about 245.3 mg of fosaprepitant dimeglumine;
about 0.28 mg of palonosetron hydrochloride;
60-200 mg of the human serum albumin;
50-500 mg of lactose.

In some embodiments, an overfill volume of about 2% to 20% is required in the production process of the lyophilized formulation. In some embodiments, an overfill volume of about 5% to 10% is required in the production process of the lyophilized formulation. In some embodiments, an overfill volume of about 5% is required in the production process of the lyophilized formulation. In some embodiments, an overfill volume of about 10% is required in the production process of the lyophilized formulation. In some embodiments, an overfill volume of about 15% is required in the production process of the lyophilized formulation. In some embodiments, an overfill volume of about 20% is required in the production process of the lyophilized formulation.

In some embodiments, the lyophilized formulation is a lyophilized formulation comprising fosaprepitant or the pharmaceutically acceptable salt thereof, palonosetron or the pharmaceutically acceptable salt thereof, the human serum albumin, and mannitol. The amounts of the components in each vial of the formulation are as follows:
220-270 mg of fosaprepitant dimeglumine;
0.25-0.31 mg of palonosetron hydrochloride;
30-300 mg of the human serum albumin;
10-600 mg of mannitol.

In some embodiments, the amounts of the components in each vial of the formulation are preferably as follows:
220-270 mg of fosaprepitant dimeglumine;
0.25-0.31 mg of palonosetron hydrochloride;
60-200 mg of the human serum albumin;
50-500 mg of mannitol.

In some embodiments, the amounts of the components in each vial of the formulation are as follows:
230-260 mg of fosaprepitant dimeglumine;
0.26-0.30 mg of palonosetron hydrochloride;
100-140 mg of the human serum albumin;
100-400 mg of mannitol.

In some embodiments, each vial of the lyophilized formulation comprises the following components:
about 245.3 mg of fosaprepitant dimeglumine;
about 0.28 mg of palonosetron hydrochloride;
30-300 mg of the human serum albumin;
10-600 mg of mannitol.

In some embodiments, an overfill volume of about 2% to 20% is required in the production process of the lyophilized formulation. In some embodiments, an overfill volume of about 5% to 10% is required in the production process of the lyophilized formulation. In some embodiments, an overfill volume of about 5% is required in the production process of the lyophilized formulation. In some embodiments, an overfill volume of about 10% is required in the production process of the lyophilized formulation. In some embodiments, an overfill volume of about 15% is required in the production process of the lyophilized formulation. In some embodiments, an overfill volume of about 20% is required in the production process of the lyophilized formulation.

The present invention further provides a method for preparing the lyophilized formulation described above. The preparation method can be performed by using conventional process equipment and features simple process and thereby is suitable for industrial production.

In some embodiments, a proper amount of a pH adjuster is also required to be added during the preparation of the lyophilized formulation described above.

In the present invention, the pH adjuster is not limited, and the pH adjuster may be all pharmaceutically acceptable pH adjusters.

In some embodiments, the pH adjuster is an alkaline pH adjuster selected from one or more of sodium hydroxide, potassium hydroxide, sodium carbonate, sodium bicarbonate, disodium hydrogen phosphate, sodium dihydrogen phosphate, triethylamine, ethylenediamine, triethanolamine, Tris (tris(hydroxymethyl)aminomethane), arginine, lysine, histidine, glycine, or meglumine, and is preferably sodium hydroxide.

In some embodiments, the method for preparing the lyophilized formulation comprising fosaprepitant or the pharmaceutically acceptable salt thereof, the human serum albumin, and the lyophilization excipient described above comprises the following steps:
(1) dissolving fosaprepitant dimeglumine, the human serum albumin, and the lyophilization excipient in water for injection, adjusting the pH of the drug solution to a proper range by using a proper amount of a pH adjuster (e.g., sodium hydroxide), and supplementing with the water for injection to bring the volume to a total formulating amount, thus obtaining an intermediate drug solution;
(2) filtering and sterilizing the intermediate drug solution, filling, partially stoppering, and lyophilizing to obtain a product.

In some embodiments, the method for preparing the lyophilized formulation comprising fosaprepitant or the pharmaceutically acceptable salt thereof, the human serum albumin, and the lyophilization excipient described above comprises the following steps:
(1) dissolving the lyophilization excipient in water for injection to obtain a first solution;
(2) adding the human serum albumin to the first solution and uniformly stirring to obtain a second solution;
(3) under continuous stirring, adding fosaprepitant dimeglumine to the second solution to gradually dissolve fosaprepitant dimeglumine, adjusting the pH of the drug solution to a proper range by using a proper amount of sodium hydroxide, and supplementing with the water for injection to bring the volume to a total formulating amount, thus obtaining an intermediate drug solution;
(4) filtering and sterilizing the intermediate drug solution, filling, partially stoppering, and lyophilizing to obtain a product.

In some embodiments, the method for preparing the lyophilized formulation comprising fosaprepitant or the pharmaceutically acceptable salt thereof, the human serum albumin, and the lyophilization excipient described above comprises the following steps:
(1) dissolving the lyophilization excipient and the human serum albumin in water for injection to obtain a first solution;
(2) under continuous stirring, adding fosaprepitant dimeglumine to the first solution to gradually dissolve fosaprepitant dimeglumine, adjusting the pH of the drug solution to a proper range by using sodium hydroxide, and supplementing with the water for injection to bring the volume to a total formulating amount, thus obtaining an intermediate drug solution;
(3) filtering and sterilizing the intermediate drug solution, filling, partially stoppering, and lyophilizing to obtain a product.

In some embodiments, the method for preparing the lyophilized formulation comprising fosaprepitant or the pharmaceutically acceptable salt thereof, palonosetron or the pharmaceutically acceptable salt thereof, the human serum albumin, and the lyophilization excipient described above comprises the following steps:
(1) dissolving fosaprepitant dimeglumine, palonosetron hydrochloride, the human serum albumin, and the lyophilization excipient in water for injection, adjusting the pH of the drug solution to a proper range by using a proper amount of a pH adjuster (e.g., sodium hydroxide), and supplementing with the water for injection to bring the volume to a total formulating amount, thus obtaining an intermediate drug solution;
(2) filtering and sterilizing the intermediate drug solution, filling, partially stoppering, and lyophilizing to obtain a product.

In some embodiments, the method for preparing the lyophilized formulation comprising fosaprepitant or the pharmaceutically acceptable salt thereof, palonosetron or the pharmaceutically acceptable salt thereof, the human serum albumin, and the lyophilization excipient described above comprises the following steps:
(1) dissolving the lyophilization excipient in water for injection to obtain a first solution;
(2) adding the human serum albumin and palonosetron hydrochloride to the first solution and uniformly stirring to obtain a second solution;
(3) under continuous stirring, adding fosaprepitant dimeglumine to the second solution to gradually dissolve fosaprepitant dimeglumine, adjusting the pH of the drug solution to a proper range by using sodium hydroxide, and supplementing with the water for injection to bring the volume to a total formulating amount, thus obtaining an intermediate drug solution;
(4) filtering and sterilizing the intermediate drug solution, filling, partially stoppering, and lyophilizing to obtain a product.

In some embodiments, the method for preparing the lyophilized formulation comprising fosaprepitant or the pharmaceutically acceptable salt thereof, palonosetron or the pharmaceutically acceptable salt thereof, the human serum albumin, and the lyophilization excipient described above comprises the following steps:
(1) dissolving the lyophilization excipient, the human serum albumin, and palonosetron hydrochloride in water for injection to obtain a first solution;
(2) under continuous stirring, adding fosaprepitant dimeglumine to the first solution to gradually dissolve fosaprepitant dimeglumine, adjusting the pH of the drug solution to a proper range by using sodium hydroxide, and supplementing with the water for injection to bring the volume to a total formulating amount, thus obtaining an intermediate drug solution;
(3) filtering and sterilizing the intermediate drug solution, filling, partially stoppering, and lyophilizing to obtain a product.

In some embodiments, the pH of the intermediate drug solution is adjusted to be in the range of 7.5 to 10.0 by adding a pH adjuster during the preparation of the lyophilized formulation described above.

In some embodiments, the pH of the intermediate drug solution is preferably adjusted to be in the range of 8.0 to 9.5 by adding a pH adjuster during the preparation of the lyophilized formulation described above.

In some embodiments, the pH of the intermediate drug solution is further preferably adjusted to be in the range of 8.5 to 9.0 by adding a pH adjuster during the preparation of the lyophilized formulation described above.

The present invention further provides a method for clinical use of the lyophilized formulation described above, which comprises the following steps:
(1) slowly injecting 5 mL of a solvent into the product along the vial wall with a syringe and slightly shaking the vial to assist dissolving;
(2) after lyophilized powder is completely dissolved, drawing a drug solution obtained by reconstitution with a syringe (5 mL) and injecting into an infusion bag or an infusion bottle containing 145 mL of a solvent, and slightly turning the infusion bag or the infusion bottle by hand 3 to 5 times to uniformly mix the drug solution, thus obtaining a drug solution for clinical infusion.

In some embodiments, the solvent in the method for clinical use of a lyophilized formulation described above comprises one of sterile water for injection, 5% glucose injection, 0.9% sodium chloride injection, and 5% glucose and sodium chloride injection, and is preferably 0.9% sodium chloride injection.

In some embodiments, the lyophilized formulation described above is a white or off-white lyophilized lump or powder. In some embodiments, the lyophilized formulation described above is a light yellow lyophilized lump or powder.

In some embodiments, the reconstitution time of the lyophilized formulation described above is no more than 30 min. In some embodiments, the reconstitution time of the lyophilized formulation described above is no more than 25 min. In some embodiments, the reconstitution time of the lyophilized formulation described above is no more than 20 min. In some embodiments, the reconstitution time of the lyophilized formulation described above is no more than 15 min. In some embodiments, the reconstitution time of the lyophilized formulation described above is no more than 10 min. In some embodiments, the reconstitution time of the lyophilized formulation described above is no more than 5 min. In some embodiments, the reconstitution time of the lyophilized formulation described above is no more than 3 min. In some embodiments, the reconstitution time of the lyophilized formulation described above is no more than 2 min. In some embodiments, the reconstitution time of the lyophilized formulation described above is no more than 1 min.

In some embodiments, the pH of the drug solution obtained by reconstitution of the lyophilized formulation described above is 7.5-10.0. In some embodiments, the pH of the drug solution obtained by reconstitution of the lyophilized formulation described above is 7.5-9.5. In some embodiments, the pH of the drug solution obtained by reconstitution of the lyophilized formulation described above is 8.0-9.5. In some embodiments, the pH of the drug solution obtained by reconstitution of the lyophilized formulation described above is 8.0-9.0. In some embodiments, the pH of the drug solution obtained by reconstitution of the lyophilized formulation described above is 8.5-9.5. In some embodiments, the pH of the lyophilized formulation described above after being reconstituted and diluted to be a drug solution for clinical infusion is 8.5-9.0. In some embodiments, the pH of the drug solution obtained by reconstitution of the lyophilized formulation described above is 8.0-8.5.

In some embodiments, the lyophilized formulation described above is a clear solution after being reconstituted and diluted to be a drug solution for clinical infusion.

In some embodiments, the pH of the lyophilized formulation described above after being reconstituted and diluted to be a drug solution for clinical infusion is 7.0-9.0. In some embodiments, the pH of the drug solution obtained by reconstitution of the lyophilized formulation described above is 7.0-8.5. In some embodiments, the pH of the drug solution obtained by reconstitution of the lyophilized formulation described above is 7.5-9.0. In some embodiments, the pH of the drug solution obtained by reconstitution of the lyophilized formulation described above is 7.5-8.5. In some embodiments, the pH of the drug solution obtained by reconstitution of the lyophilized formulation described above is 8.0-8.5. In some embodiments, the pH of the lyophilized formulation described above after being reconstituted and diluted to be a drug solution for clinical infusion is 7.5-8.0.

In some embodiments, the lyophilized formulation described above remains to be a clear solution for at least 1 h after being reconstituted and diluted to be a drug solution for clinical infusion. In some embodiments, the lyophilized formulation described above remains to be a clear solution for at least 2 h after being reconstituted and diluted to be a drug solution for clinical infusion. In some embodiments, the lyophilized formulation described above remains to be a clear solution for at least 3 h after being reconstituted and diluted to be a drug solution for clinical infusion. In some embodiments, the lyophilized formulation described above remains to be a clear solution for at least 4 h after being reconstituted and diluted to be a drug solution for clinical infusion. In some embodiments, the lyophilized formulation described above remains to be a clear solution for at least 6 h after being reconstituted and diluted to be a drug solution for clinical infusion. In some embodiments, the lyophilized formulation described above remains to be a clear solution for at least 8 h after being reconstituted and diluted to be a drug solution for clinical infusion. In some embodiments, the lyophilized formulation described above remains to be a clear solution for at least 12 h after being reconstituted and diluted to be a drug solution for clinical infusion. In some embodiments, the lyophilized formulation described above remains to be a clear solution for at least 24 h after being reconstituted and diluted to be a drug solution for clinical infusion.

In some embodiments, at a temperature of about 0° C. to about 25° C., the lyophilized formulation described above remains to be a clear solution for at least 1 h after being reconstituted and diluted to be a drug solution for clinical infusion. In some embodiments, at a temperature of about 0° C. to about 25° C., the lyophilized formulation described above remains to be a clear solution for at least 2 h after being reconstituted and diluted to be a drug solution for clinical infusion. In some embodiments, at a temperature of about 0° C. to about 25° C., the lyophilized formulation described above remains to be a clear solution for at least 3 h after being reconstituted and diluted to be a drug solution for clinical infusion. In some embodiments, at a temperature of about 0° C. to about 25° C., the lyophilized formulation described above remains to be a clear solution for at least 4 h after being reconstituted and diluted to be a drug solution for clinical infusion. In some embodiments, at a temperature of about 0° C. to about 25° C., the lyophilized formulation described above remains to be a clear solution for at least 6 h after being reconstituted and diluted to be a drug solution for clinical infusion. In some embodiments, at a temperature of about 0° C. to about 25° C., the lyophilized formulation described above remains to be a clear solution for at least 8 h after being reconstituted and diluted to be a drug solution for clinical infusion. In some embodiments, at a temperature of about 0° C. to about 25° C., the lyophilized formulation described above remains to be a clear solution for at least 12 h after being reconstituted and diluted to be a drug solution for clinical infusion. In some embodiments, at a temperature of about 0° C. to about 25° C., the lyophilized formulation described above remains to be a clear solution for at least 24 h after being reconstituted and diluted to be a drug solution for clinical infusion.

In some embodiments, at room temperature, the lyophilized formulation described above remains to be a clear solution for at least 1 h after being reconstituted and diluted to be a drug solution for clinical infusion. In some embodiments, at room temperature, the lyophilized formulation described above remains to be a clear solution for at least 2 h after being reconstituted and diluted to be a drug solution for clinical infusion. In some embodiments, at room temperature, the lyophilized formulation described above remains to be a clear solution for at least 3 h after being reconstituted and diluted to be a drug solution for clinical infusion. In some embodiments, at room temperature, the lyophilized formulation described above remains to be a clear solution for at least 4 h after being reconstituted and diluted to be a drug solution for clinical infusion. In some embodiments, at room temperature, the lyophilized formulation described above remains to be a clear solution for at least 6 h after being reconstituted and diluted to be a drug solution for clinical infusion. In some embodiments, at room temperature, the lyophilized formulation described above remains to be a clear solution for at least 8 h after being reconstituted and diluted to be a drug solution for clinical infusion. In some embodiments, at room temperature, the lyophilized formulation described above remains to be a clear solution for at least 12 h after being reconstituted and diluted to be a drug solution for clinical infusion. In some embodiments, at room temperature, the lyophilized formulation described above remains to be a clear solution for at least 24 h after being reconstituted and diluted to be a drug solution for clinical infusion.

### Beneficial Effects

Compared with the prior art, the present invention can obtain the following beneficial effects:

The lyophilized formulation comprising fosaprepitant or a pharmaceutically acceptable salt thereof and human serum albumin or the lyophilized formulation comprising fosaprepitant or a pharmaceutically acceptable salt thereof, human serum albumin, and palonosetron or a pharmaceutically acceptable salt provided by the present invention takes a certain amount of human serum albumin as a solubilizer and comprises a certain amount of a lyophilization excipient and a pH adjuster, and the ratio of the drug substance to the excipients is reasonable, and solve a series of problems including long reconstitution time, generation of a large number of bubbles during reconstitution, inability to guarantee complete reconstitution within a certain time, poor storage stability of finished products, etc. The resulting lyophilized formulations feature short reconstitution time, do not result in precipitation of particles at room temperature within 24 h after being reconstituted and formulated into a drug solution for clinical use, have good storage stability, and are safe and effective; besides, the formulations are characterized by simple preparation process and excellent comprehensive performance and are suitable for industrial production.

### EMBODIMENTS OF THE PRESENT INVENTION

The present invention is further described below by means of specific examples, which are not intended to limit the scope of the present invention.

### Example 1

### 1.1 Formula Information

| Drug Substance and | Formula | | Formula | | Formula | | Formula | | Formula | |
|---|---|---|---|---|---|---|---|---|---|---|
| Excipients | 1-1 | | 1-2 | | 1-3 | | 1-4 | | 1-5 | |
| Fosaprepitant dimeglumine | 1.545 | g | 1.545 | g | 1.545 | g | 1.545 | g | 1.545 | g |
| Human serum albumin | 240 | mg | 360 | mg | 600 | mg | 1200 | mg | 2400 | mg |
| Lyophilization excipient | 0 | mg | 0 | mg | 0 | mg | 0 | mg | 0 | mg |
| 0.5 mol/L sodium | q.s. | | q.s. | | q.s. | | q.s. | | q.s. | |
| hydroxide | | | | | | | | | | |
| Purified water | To 36 | mL | To 36 | mL | To 36 | mL | To 36 | mL | To 36 | mL |

### 1.2 Preparation Process

A proper amount of water (10-30° C.) and human serum albumin at an amount specified in the formula were added to a 100 mL beaker (containing a stir bar), and then the stirring was started. The total amount of the solution in this step was 88.33% of the formulating amount.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 5 vials in each batch. The vials were partially stoppered, transferred to a lyophilizer, and lyophilized for 30 h to obtain finished products.

### 1.3 Reconstitution Process

One vial in each of the above 5 batches of lyophilized samples and a proper amount of 0.9% sodium chloride injection were left to stand to reach room temperature.

5 mL of 0.9% sodium chloride injection was drawn with a syringe (5 mL) and slowly injected into each of the 5 vials of samples along the wall to reconstitute the samples, and the reconstitution time was recorded.

### 1.4 Results of Reconstitution Experiment

| | | Amount of | | | | |
|---|---|---|---|---|---|---|
| | | human | | | | |
| | Amount of | serum | | | Number of | |
| | lyophilization | albumin | | | Bubbles | |
| Sample | excipient | specified | | | Generated | |
| (Formula | specified in | in the | | Reconstitution | During | Influence of |
| No.) | the formula | formula | | Time (min) | Reconstitution | Bubbles |
| Formula 1-1 | 0 mg/vial | 40 | mg/vial | 2 | + | Does not |
| | | | | | | influence |
| | | | | | | reconstitution |
| Formula 1-2 | 0 mg/vial | 60 | mg/vial | 2.5 | ++ | Does not |
| | | | | | | influence |
| | | | | | | reconstitution |
| Formula 1-3 | 0 mg/vial | 100 | mg/vial | 10 | +++ | Does not |
| | | | | | | influence |
| | | | | | | reconstitution |
| Formula 1-4 | 0 mg/vial | 200 | mg/vial | 17 | ++++ | Has little |
| | | | | | | influence on |
| | | | | | | reconstitution |
| Formula 1-5 | 0 mg/vial | 400 | mg/vial | 25 | +++ | Does not |
| | | | | | | dissipate after a |
| | | | | | | long time (no |
| | | | | | | less than 2 h) |
| | | | | | | and has great |
| | | | | | | influence on |
| | | | | | | reconstitution |

Note:
more “+” sign indicates more bubbles generated during reconstitution.

### 1.5 Conclusion

This example compares the influences of different amounts of human serum albumin in the formula on reconstitution time, in the absence of a lyophilization excipient. As can be seen from the results, the reconstitution time of the formulation significantly increased with the increase of the amount of human serum albumin specified in the formula, and the number of bubbles generated during the reconstitution process also significantly increased with the increase of the amount of human serum albumin specified in the formula, wherein when the amount of human serum albumin specified in the formula was 400 mg/vial, a large number of bubbles were generated and did not dissipate after a long time (no less than 2 h), which had great influence on reconstitution.

### Example 2

### 2.1 Formula Information

| Drug Substance and | | | | | | |
|---|---|---|---|---|---|---|
| Excipients | Formula 2-1 | | Formula 2-2 | | Formula 2-3 | |
| Fosaprepitant dimeglumine | 1.0303 | g | 1.0303 | g | 1.0303 | g |
| Human serum albumin | 1600 | mg | 2400 | mg | 3200 | mg |
| Lyophilization excipient | 0 | mg | 0 | mg | 0 | mg |
| 0.5 mol/L sodium hydroxide | q.s. | | q.s. | | q.s. | |
| Purified water | To 24 | mL | To 24 | mL | To 24 | mL |

### 2.2 Preparation Process

A proper amount of water (10-30° C.) and human serum albumin at an amount specified in the formula were added to a 50 mL beaker (containing a stir bar), and then the stirring was started. The total amount of the solution in this step was 88.33% of the formulating amount.

Fosaprepitant dimeglumine at an amount specified in the formula was weighed out and added to the above 50 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 3 vials in each batch. The vials were partially stoppered, transferred to a lyophilizer, and lyophilized for 30 h to obtain finished products.

### 2.3 Reconstitution Process

One vial in each of the above 3 batches of lyophilized samples and a proper amount of 0.9% sodium chloride injection were left to stand to reach room temperature.

5 mL of 0.9% sodium chloride injection was drawn with a syringe (5 mL) and slowly injected into each of the 3 vials of samples along the wall to reconstitute the samples, and the reconstitution time was recorded.

### 2.4 Results of Reconstitution Experiment

| | | Amount | | | |
|---|---|---|---|---|---|
| | | of human | | | |
| | Amount of | serum | | Number of | |
| | lyophilization | albumin | | Bubbles | |
| Sample | excipient | specified | | Generated | |
| (Formula | specified in | in the | Reconstitution | During | Influence of |
| No.) | the formula | formula | Time (min) | Reconstitution | Bubbles |
| Formula 2-1 | 0 mg/vial | 400 mg/vial | 30 | +++ | Does not |
| | | | | | dissipate after a |
| | | | | | long time (no less |
| | | | | | than 2 h) and has |
| | | | | | great influence on |
| | | | | | reconstitution |
| Formula 2-2 | 0 mg/vial | 600 mg/vial | 56 | + | Does not |
| | | | | | dissipate after a |
| | | | | | long time (no less |
| | | | | | than 2 h) and has |
| | | | | | great influence on |
| | | | | | reconstitution |
| Formula 2-3 | 0 mg/vial | 800 mg/vial | Hasn't | | Does not |
| | | | dissolved | | dissipate after a |
| | | | completely in | | long time (no less |
| | | | 1 h | | than 2 h) and |
| | | | | | poses the risk of |
| | | | | | incomplete |
| | | | | | reconstitution |

Note:
more “+” sign indicates more bubbles generated during reconstitution.

### 2.5 Conclusion

This example compares the influences of different amounts of human serum albumin in the formula on reconstitution time, in the absence of a lyophilization excipient. The results indicate that the reconstitution time of the formulation significantly increased with the increase of the amount of human serum albumin specified in the formula, and the number of bubbles generated during the reconstitution process also significantly increased with the increase of the amount of human serum albumin specified in the formula, wherein when the amount of human serum albumin specified in the formula was greater than 400 mg/vial, the reconstitution time was no less than 30 min, and a large number of bubbles were generated and did not dissipate after a long time (no less than 2 h), which had a great influence on reconstitution and even posed the risk of incomplete reconstitution.

### Example 3

### 3.1 Formula Information

| Drug Substance and | Formula | | Formula | | Formula | | Formula | | Formula | |
|---|---|---|---|---|---|---|---|---|---|---|
| Excipients | 3-1 | | 3-2 | | 3-3 | | 3-4 | | 3-5 | |
| Fosaprepitant dimeglumine | 1.545 | g | 1.545 | g | 1.545 | g | 1.545 | g | 1.545 | g |
| Palonosetron hydrochloride | 1.764 | mg | 1.764 | mg | 1.764 | mg | 1.764 | mg | 1.764 | mg |
| Human serum albumin | 360 | mg | 480 | mg | 600 | mg | 1200 | mg | 2400 | mg |
| Lyophilization excipient | 0 | mg | 0 | mg | 0 | mg | 0 | mg | 0 | mg |
| 0.5 mol/L sodium | q.s. | | q.s. | | q.s. | | q.s. | | q.s. | |
| hydroxide | | | | | | | | | | |
| Purified water | To 36 | mL | To 36 | mL | To 36 | mL | To 36 | mL | To 36 | mL |

### 3.2 Preparation Process

A proper amount of water (10-30° C.) and human serum albumin and palonosetron hydrochloride each at an amount specified in the formula were added to a 100 ml beaker (containing a stir bar), and then the stirring was started. The total amount of the solution in this step was 88.33% of the formulating amount.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 ml vials at an amount of 6 mL per vial. Each formula was 1 batch, and there were 5 vials in each batch. The vials were partially stoppered, transferred to a lyophilizer, and lyophilized for 30 h to obtain finished products.

### 3.3 Reconstitution Process

One vial in each of the above 5 batches of lyophilized samples and a proper amount of 0.9% sodium chloride injection were left to stand to reach room temperature.

5 mL of 0.9% sodium chloride injection was drawn with a syringe (5 mL) and slowly injected into each of the 5 vials of samples along the wall to reconstitute the samples, and the reconstitution time was recorded.

### 3.4 Results of Reconstitution Experiment

| | | Amount of | | | | |
|---|---|---|---|---|---|---|
| | | human | | | | |
| | Amount of | serum | | | Number of | |
| | lyophilization | albumin | | | Bubbles | |
| Sample | excipient | specified | | | Generated | |
| (Formula | specified in the | in the | | Reconstitution | During | Influence of |
| No.) | formula | formula | | Time (min) | Reconstitution | Bubbles |
| Formula | 0 mg/vial | 60 | mg/vial | 2.5 | + | Does not |
| 3-1 | | | | | | influence |
| | | | | | | reconstitution |
| Formula | 0 mg/vial | 80 | mg/vial | 7 | ++ | Does not |
| 3-2 | | | | | | influence |
| | | | | | | reconstitution |
| Formula | 0 mg/vial | 100 | mg/vial | 11 | +++ | Does not |
| 3-3 | | | | | | influence |
| | | | | | | reconstitution |
| Formula | 0 mg/vial | 200 | mg/vial | 17 | ++++ | Has little |
| 3-4 | | | | | | influence on |
| | | | | | | reconstitution |
| Formula | 0 mg/vial | 400 | mg/vial | 30 | ++++ | Does not |
| 3-5 | | | | | | dissipate after |
| | | | | | | a long time |
| | | | | | | (no less than 2 |
| | | | | | | h) and has |
| | | | | | | great |
| | | | | | | influence on |
| | | | | | | reconstitution |

Note:
more “+” sign indicates more bubbles generated during reconstitution.

### 3.5 Conclusion

This example further compares, for a compound formulation comprising palonosetron hydrochloride, the influences of different amounts of human serum albumin in the formula on reconstitution time in the absence of a lyophilization excipient. The results indicate that, similar to the rule of the single-drug formulation in Example 1, the reconstitution time of the compound formulation of this example also significantly increased with the increase of the amount of human serum albumin specified in the formula, and the number of bubbles generated during the reconstitution process also significantly increased with the increase of the amount of human serum albumin specified in the formula, wherein when the amount of human serum albumin specified in the formula was 400 mg/vial, a large number of bubbles were generated and did not dissipate after a long time (no less than 2 h), which had great influence on reconstitution.

### Example 4

### 4.1 Formula Information

| Drug Substance and | | | | | | |
|---|---|---|---|---|---|---|
| Excipients | Formula 4-1 | | Formula 4-2 | | Formula 4-3 | |
| Fosaprepitant dimeglumine | 1.0303 | g | 1.0303 | g | 1.0303 | g |
| Palonosetron hydrochloride | 1.176 | mg | 1.176 | mg | 1.176 | mg |
| Human serum albumin | 1600 | mg | 2400 | mg | 3200 | mg |
| Lyophilization excipient | 0 | mg | 0 | mg | 0 | mg |
| 0.5 mol/L sodium hydroxide | q.s. | | q.s. | | q.s. | |
| Purified water | To 24 | mL | To 24 | mL | To 24 | mL |

### 4.2 Preparation Process

A proper amount of water (10-30° C.) and human serum albumin and palonosetron hydrochloride each at an amount specified in the formula were added to a 50 mL beaker (containing a stir bar), and then the stirring was started. The total amount of the solution in this step was 88.33% of the formulating amount.

Fosaprepitant dimeglumine at an amount specified in the formula was weighed out and added to the above 50 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 3 vials in each batch. The vials were partially stoppered, transferred to a lyophilizer, and lyophilized for 30 h to obtain finished products.

### 4.3 Reconstitution Process

One vial in each of the above 3 batches of lyophilized samples and a proper amount of 0.9% sodium chloride injection were left to stand to reach room temperature.

5 mL of 0.9% sodium chloride injection was drawn with a syringe (5 mL) and slowly injected into each of the 3 vials of samples along the wall to reconstitute the samples, and the reconstitution time was recorded.

### 4.4 Results of Reconstitution Experiment

| | | Amount | | | |
|---|---|---|---|---|---|
| | | of human | | | |
| | Amount of | serum | | Number of | |
| | lyophilization | albumin | | Bubbles | |
| Sample | excipient | specified | | Generated | |
| (Formula | specified in | in the | Reconstitution | During | |
| No.) | the formula | formula | Time (min) | Reconstitution | Influence of Bubbles |
| Formula | 0 mg/vial | 400 mg/vial | 34 | ++++++ | Does not dissipate after |
| 4-1 | | | | | a long time (no less |
| | | | | | than 2 h) and has great |
| | | | | | influence on |
| | | | | | reconstitution |
| Formula | 0 mg/vial | 600 mg/vial | 60 | ++++++++ | Does not dissipate after |
| 4-2 | | | | | a long time (no less |
| | | | | | than 2 h) and has great |
| | | | | | influence on |
| | | | | | reconstitution |
| Formula | 0 mg/vial | 800 mg/vial | Hasn't dissolved | ++++++++++ | Does not dissipate after |
| 4-3 | | | completely in 1 h | | a long time (no less |
| | | | | | than 2 h) and poses the |
| | | | | | risk of incomplete |
| | | | | | reconstitution |

Note:
more “+” sign indicates more bubbles generated during reconstitution.

### 4.5 Conclusion

This example further compares, for a compound formulation comprising palonosetron hydrochloride, the influences of different amounts of human serum albumin in the formula on reconstitution time in the absence of a lyophilization excipient. The results indicate that, the reconstitution time of the formulation significantly increased with the increase of the amount of human serum albumin specified in the formula, and the number of bubbles generated during the reconstitution process also significantly increased with the increase of the amount of human serum albumin specified in the formula, wherein when the amount of human serum albumin specified in the formula was greater than 400 mg/vial, the reconstitution time was no less than 30 min, and a large number of bubbles were generated and did not dissipate after a long time (no less than 2 h), which had a great influence on reconstitution and even posed the risk of incomplete reconstitution.

### Example 5

### 5.1 Formula Information

| Drug Substance and | | | | | | | | |
|---|---|---|---|---|---|---|---|---|
| Excipients | Formula 5-1 | | Formula 5-2 | | Formula 5-3 | | Formula 5-4 | |
| Fosaprepitant dimeglumine | 1.545 | g | 1.545 | g | 1.545 | g | 1.545 | g |
| Human serum albumin | 600 | mg | 1200 | mg | 1800 | mg | 2400 | mg |
| Lyophilization excipient | 0 | mg | 0 | mg | 0 | mg | 0 | mg |
| 0.5 mol/L sodium hydroxide | q.s. | | q.s. | | q.s. | | q.s. | |
| Purified water | To 36 | mL | To 36 | mL | To 36 | mL | To 36 | mL |

### 5.2 Preparation Process

A proper amount of water (10-30° C.) and human serum albumin at an amount specified in the formula were added to a 100 mL beaker (containing a stir bar), and then the stirring was started. The total amount of the solution in this step was 88.33% of the formulating amount.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 5 vials in each batch. The vials were partially stoppered, transferred to a lyophilizer, and lyophilized for 30 h to obtain finished products.

### 5.3 Reconstitution Process

One vial in each of the above 4 batches of lyophilized samples and a proper amount of 0.9% sodium chloride injection were left to stand to reach room temperature.

5 mL of 0.9% sodium chloride injection was drawn with a syringe (5 mL) and slowly injected into each of the 4 vials of samples along the wall to reconstitute the samples, and the reconstitution time was recorded.

The reconstituted samples were each drawn with a syringe (5 mL) and injected into an infusion bottle containing 145 mL of 0.9% sodium chloride injection to be diluted to 150 mL, the infusion bottle was slightly turned 3 times to uniformly mix the drug solution, and the drug solution was left to stand at room temperature and observed whether visible particles were precipitated from the drug solution.

### 5.4 Results of Reconstitution Experiment

| | | Amount of | | |
|---|---|---|---|---|
| | Amount of | human serum | | |
| Sample | lyophilization | albumin | | |
| (Formula | excipient specified | specified in the | Reconstitution | Observation of Visible |
| No.) | in the formula | formula | Time (min) | Particles |
| Formula | 0 mg/vial | 100 mg/vial | 11 | Being clear and free of |
| 5-1 | | | | visible particles after |
| | | | | being left to stand at |
| | | | | room temperature for 8 |
| | | | | h |
| Formula | 0 mg/vial | 200 mg/vial | 16 | Being clear and free of |
| 5-2 | | | | visible particles after |
| | | | | being left to stand at |
| | | | | room temperature for 8 |
| | | | | h |
| Formula | 0 mg/vial | 300 mg/vial | 20 | Being clear and free of |
| 5-3 | | | | visible particles after |
| | | | | being left to stand at |
| | | | | room temperature for 8 |
| | | | | h |
| Formula | 0 mg/vial | 400 mg/vial | 24 | Being clear and free of |
| 5-4 | | | | visible particles after |
| | | | | being left to stand at |
| | | | | room temperature for 8 |
| | | | | h |

### 5.5 Experiment on Storage Stability Examination and Results

The above 4 batches of lyophilized samples were subjected to stability examination at 60° C., 40° C., and 25° C. and compared, and the results are as follows:

Results of destruction experiment at 60° C.

| Related | Examination | Sample (Formula No.) | | | |
|---|---|---|---|---|---|
| Substance % | Conditions | Formula 5-1 | Formula 5-2 | Formula 5-3 | Formula 5-4 |
| Impurity C | 0 days | 0.120 | 0.147 | 0.173 | 0.188 |
| (aprepitant) | 60° C., 3 days | 2.771 | 3.658 | 4.508 | 5.204 |
| | 60° C., 5 days | 4.158 | 5.227 | 6.223 | 7.242 |
| Total | 0 days | 0.211 | 0.258 | 0.281 | 0.275 |
| impurity | 60° C., 3 days | 3.132 | 4.223 | 5.192 | 5.983 |
| | 60° C., 5 days | 4.567 | 5.812 | 6.971 | 8.061 |

Results of destruction experiment at 40° C.

| Related | Examination | Sample (Formula No.) | | | |
|---|---|---|---|---|---|
| Substance % | Conditions | Formula 5-1 | Formula 5-2 | Formula 5-3 | Formula 5-4 |
| Impurity C | 0 days | 0.120 | 0.147 | 0.173 | 0.188 |
| (aprepitant) | 40° C., 5 days | 0.722 | 1.042 | 1.336 | 1.570 |
| | 40° C., 11 days | 1.103 | 1.565 | 2.048 | 2.367 |
| | 40° C., 15 days | 1.383 | 1.933 | 2.466 | 2.863 |
| Total | 0 days | 0.211 | 0.258 | 0.281 | 0.275 |
| impurity | 40° C., 5 days | 0.892 | 1.285 | 1.674 | 1.989 |
| | 40° C., 11 days | 1.335 | 1.932 | 2.529 | 2.942 |
| | 40° C., 15 days | 1.689 | 2.381 | 3.053 | 3.571 |

Results of accelerated experiment at 25° C.

| Related | Examination | Sample (Formula No.) | | | |
|---|---|---|---|---|---|
| Substance % | Conditions | Formula 5-1 | Formula 5-2 | Formula 5-3 | Formula 5-4 |
| Impurity C | 0 days | 0.120 | 0.147 | 0.173 | 0.188 |
| (aprepitant) | 25° C., 1 | 0.468 | 0.667 | 0.811 | 0.985 |
| | month | | | | |
| | 25° C., 2 | 0.652 | 0.942 | 1.172 | 1.388 |
| | months | | | | |
| | 25° C., 3 | 0.871 | 1.268 | 1.599 | 1.893 |
| | months | | | | |
| | 25° C., 6 | 1.200 | 1.725 | 2.190 | 2.564 |
| | months | | | | |
| Total | 0 days | 0.211 | 0.258 | 0.281 | 0.275 |
| impurity | 25° C., 1 | 0.561 | 0.801 | 1.006 | 1.230 |
| | month | | | | |
| | 25° C., 2 | 0.778 | 1.155 | 1.451 | 1.708 |
| | months | | | | |
| | 25° C., 3 | 1.049 | 1.526 | 1.942 | 2.268 |
| | months | | | | |
| | 25° C., 6 | 1.448 | 2.055 | 2.634 | 3.065 |
| | months | | | | |

### 5.6 Conclusion

This example compares the influences of different amounts of human serum albumin in the formula on reconstitution time, stability of the drug solution obtained by reconstitution, and storage stability of formulation, in the absence of a lyophilization excipient. As can be seen from the reconstitution results, the reconstitution time of the formulation significantly increased with the increase of the amount of human serum albumin specified in the formula, and the drug solution obtained by reconstitution was able to remain stable for no less than 8 h. As can be seen from the results of stability examination, the growth rate of the related substances significantly accelerated with the increase of the amount of human serum albumin specified in the formula, and the storage stability of the formulation significantly decreased with the increase of the amount of human serum albumin specified in the formula.

### Example 6

### 6.1 Formula information

| Drug Substance and | | | | | | | | |
|---|---|---|---|---|---|---|---|---|
| Excipients | Formula 6-1 | | Formula 6-2 | | Formula 6-3 | | Formula 6-4 | |
| Fosaprepitant dimeglumine | 1.545 | g | 1.545 | g | 1.545 | g | 1.545 | g |
| Palonosetron hydrochloride | 1.764 | mg | 1.764 | mg | 1.764 | mg | 1.764 | mg |
| Human serum albumin | 240 | mg | 600 | mg | 1200 | mg | 1800 | mg |
| Lyophilization excipient | 0 | mg | 0 | mg | 0 | mg | 0 | mg |

| | | | | |
|---|---|---|---|---|
| 0.5 mol/L sodium hydroxide | q.s. | q.s. | q.s. | q.s. |
| Purified water | To 36 mL | To 36 mL | To 36 mL | To 36 mL |

### 6.2 Preparation Process

A proper amount of water (10-30° C.) and palonosetron hydrochloride at an amount specified in the formula were added to a 100 ml beaker (containing a stir bar), and then the stirring was started, followed by addition of human serum albumin at an amount specified in the formula. The total amount of the solution in this step was 88.33% of the formulating amount.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 5 vials in each batch. The vials were partially stoppered, transferred to a lyophilizer, and lyophilized for 30 h to obtain finished products.

### 6.3 Reconstitution Process

One vial in each of the above 4 batches of lyophilized samples and a proper amount of 0.9% sodium chloride injection were left to stand to reach room temperature.

5 mL of 0.9% sodium chloride injection was drawn with a syringe (5 mL) and slowly injected into each of the 4 vials of samples along the wall to reconstitute the samples.

The reconstituted samples were each drawn with a syringe (5 mL) and injected into an infusion bottle containing 145 mL of 0.9% sodium chloride injection to be diluted to 150 mL, the infusion bottle was slightly turned 3 times to uniformly mix the drug solution, and the drug solution was left to stand at room temperature and observed whether visible particles were precipitated from the drug solution.

### 6.4 Results of Reconstitution Experiment

| | Amount of | Amount of | | |
|---|---|---|---|---|
| | lyophilization | human serum | | |
| Sample | excipient | albumin | | |
| (Formula | specified in the | specified in the | Reconstitution | Observation of Visible |
| No.) | formula | formula | Time (min) | Particles |
| Formula 6-1 | 0 mg/vial | 40 mg/vial | 1.5 | Precipitating a large |
| | | | | number of particles after |
| | | | | being left to stand at |
| | | | | room temperature for 15 |
| | | | | min |
| Formula 6-2 | 0 mg/vial | 100 mg/vial | 14 | Being clear and free of |
| | | | | visible particles after |
| | | | | being left to stand at |
| | | | | room temperature for 8 h |
| Formula 6-3 | 0 mg/vial | 200 mg/vial | 18 | Being clear and free of |
| | | | | visible particles after |
| | | | | being left to stand at |
| | | | | room temperature for 8 h |
| Formula 6-4 | 0 mg/vial | 300 mg/vial | 21 | Being clear and free of |
| | | | | visible particles after |
| | | | | being left to stand at |
| | | | | room temperature for 8 h |

### 6.5 Experiment on Storage Stability Examination and Results

The above 4 batches of lyophilized samples were subjected to stability examination at 40° C. and 25° C. and compared, and the results are as follows:

Results of destruction experiment at 40° C.

| Related | Examination | Sample (Formula No.) | | | |
|---|---|---|---|---|---|
| Substance % | Conditions | Formula 6-1 | Formula 6-2 | Formula 6-3 | Formula 6-4 |
| Impurity C | 0 days | 0.176 | 0.199 | 0.228 | 0.257 |
| (aprepitant) | 40° C., 5 days | 0.630 | 1.200 | 0.838 | 1.598 |
| | 40° C., 12 days | 1.041 | 1.317 | 1.877 | 2.501 |
| | 40° C., 15 days | 1.176 | 1.490 | 2.093 | 2.804 |
| Total | 0 days | 0.299 | 0.329 | 0.383 | 0.436 |
| impurity | 40° C., 5 days | 0.754 | 1.522 | 1.038 | 2.007 |
| | 40° C., 12 days | 1.191 | 1.554 | 2.234 | 3.000 |
| | 40° C., 15 days | 1.313 | 1.715 | 2.449 | 3.309 |

Results of accelerated experiment at 25° C.

| Related | Examination | Sample (Formula No.) | | | |
|---|---|---|---|---|---|
| Substance % | Conditions | Formula 6-1 | Formula 6-2 | Formula 6-3 | Formula 6-4 |
| Impurity C | 0 days | 0.176 | 0.199 | 0.228 | 0.257 |
| (aprepitant) | 25° C., 1 month | 0.420 | 0.552 | 0.760 | 0.994 |
| | 25° C., 2 months | / | 0.680 | 0.939 | 1.251 |
| | 25° C., 4 months | 0.782 | 1.066 | 1.505 | 2.031 |
| Total | 0 days | 0.299 | 0.329 | 0.383 | 0.436 |
| impurity | 25° C., 1 month | 0.531 | 0.700 | 0.955 | 1.250 |
| | 25° C., 2 months | / | 0.860 | 1.222 | 1.617 |
| | 25° C., 4 months | 0.919 | 1.256 | 1.822 | 2.400 |

### 6.6 Conclusion

This example further compares, for a compound formulation comprising palonosetron hydrochloride, the influences of different amounts of human serum albumin in the formula on reconstitution time, stability of the drug solution obtained by reconstitution, and storage stability of formulation, in the absence of a lyophilization excipient. The results indicate that the reconstitution time of the formulation of this example significantly increased with the increase of the amount of human serum albumin specified in the formula. The results of stability examination indicate that, similar to the rule of the single-drug formulation in Example 3, the storage stability of the compound formulation of this example significantly decreased with the increase of the amount of human serum albumin specified in the formula.

### Example 7

### 7.1 Formula Information

| Drug Substance and | | | | | | | | |
|---|---|---|---|---|---|---|---|---|
| Excipients | Formula 7-1 | | Formula 7-2 | | Formula 7-3 | | Formula 7-4 | |
| Fosaprepitant dimeglumine | 1.545 | g | 1.545 | g | 1.545 | g | 1.545 | g |
| Human serum albumin | 600 | mg | 600 | mg | 600 | mg | 600 | mg |
| Lactose | 0 | mg | 600 | mg | 1200 | mg | 1800 | mg |

| | | | | |
|---|---|---|---|---|
| 0.5 mol/L sodium hydroxide | q.s. | q.s. | q.s. | q.s. |
| Purified water | To 36 mL | To 36 mL | To 36 mL | To 36 mL |

### 7.2 Preparation Process

Lactose at an amount specified in the formula was weighed out and added to a 100 mL beaker (containing a stir bar), water (20-60° C.) that was 80% of the formulating amount was added, and then the stirring was started to dissolve lactose.

The drug solution was cooled to 10-30° C., and human serum albumin at an amount specified in the formula was weighed out and added to the above 100 ml beaker.

Fosaprepitant dimeglumine at an amount specified in the formula was weighed out and added to the above 100 ml beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. The vials were partially stoppered, transferred to a lyophilizer, and lyophilized for 30 h to obtain finished products.

### 7.3 Reconstitution Process

One vial in each of the above 4 batches of lyophilized samples and a proper amount of 0.9% sodium chloride injection were left to stand to reach room temperature.

5 mL of 0.9% sodium chloride injection was drawn with a syringe (5 mL) and slowly injected into each of the 4 vials of samples along the wall to reconstitute the samples.

The reconstituted samples were each drawn with a syringe (5 mL) and injected into an infusion bottle containing 145 mL of 0.9% sodium chloride injection to be diluted to 150 mL, the infusion bottle was slightly turned 3 times to uniformly mix the drug solution, and the drug solution was left to stand at room temperature and observed whether visible particles were precipitated from the drug solution.

### 7.4 Results of Reconstitution Experiment

| | | Amount of | | |
|---|---|---|---|---|
| | Amount of | human serum | | |
| Sample | Lactose | albumin | | |
| (Formula | specified in | specified in the | Reconstitution | Observation of Visible |
| No.) | the formula | formula | Time (min) | Particles |
| Formula 7-1 | 0 mg/vial | 100 mg/vial | 12 | Being clear and free of visible |
| | | | | particles after being left to |
| | | | | stand at room temperature for |
| | | | | 8 h |
| Formula 7-2 | 100 mg/vial | 100 mg/vial | 9 | Being clear and free of visible |
| | | | | particles after being left to |
| | | | | stand at room temperature for |
| | | | | 8 h |
| Formula 7-3 | 200 mg/vial | 100 mg/vial | 8 | Being clear and free of visible |
| | | | | particles after being left to |
| | | | | stand at room temperature for |
| | | | | 8 h |
| Formula 7-4 | 300 mg/vial | 100 mg/vial | 3 | Being clear and free of visible |
| | | | | particles after being left to |
| | | | | stand at room temperature for |
| | | | | 8 h |

### 7.5 Experiment on Storage Stability Examination and Results

The above 4 batches of lyophilized samples were subjected to stability examination at 40° C. and 25° C. and compared, and the results are as follows:

Results of destruction experiment at 40° C.

| Related | Examination | Sample (Formula No.) | | | |
|---|---|---|---|---|---|
| Substance % | Conditions | Formula 7-1 | Formula 7-2 | Formula 7-3 | Formula 7-4 |
| Impurity C | 0 days | 0.100 | 0.090 | 0.085 | 0.085 |
| (aprepitant) | 40° C., 5 days | 0.790 | 0.570 | 0.488 | 0.459 |
| | 40° C., 10 days | 1.176 | 0.866 | 0.679 | 0.629 |
| | 40° C., 14 days | 1.43 | 0.970 | 0.800 | 0.730 |
| Total | 0 days | 0.178 | 0.176 | 0.202 | 0.188 |
| impurity | 40° C., 5 days | 0.949 | 0.713 | 0.565 | 0.526 |
| | 40° C., 10 days | 1.386 | 0.982 | 0.758 | 0.704 |
| | 40° C., 14 days | 1.670 | 1.120 | 0.930 | 0.850 |

Results of accelerated experiment at 25° C.

| Related | Examination | Sample (Formula No.) | | | |
|---|---|---|---|---|---|
| Substance % | Conditions | Formula 7-1 | Formula 7-2 | Formula 7-3 | Formula 7-4 |
| Impurity C | 0 days | 0.100 | 0.090 | 0.085 | 0.085 |
| (aprepitant) | 25° C., 1 | 0.485 | 0.370 | 0.315 | 0.303 |
| | month | | | | |
| | 25° C., 2 | 0.686 | 0.519 | 0.442 | 0.412 |
| | months | | | | |
| | 25° C., 3 | 0.911 | 0.683 | 0.586 | 0.550 |
| | months | | | | |
| Total | 0 days | 0.178 | 0.176 | 0.202 | 0.188 |
| impurity | 25° C., 1 | 0.667 | 0.511 | 0.441 | 0.421 |
| | month | | | | |
| | 25° C., 2 | 0.856 | 0.634 | 0.544 | 0.506 |
| | months | | | | |
| | 25° C., 3 | 1.050 | 0.784 | 0.673 | 0.634 |
| | months | | | | |

### 7.6 Conclusion

This example compares the influences of different amounts of lactose in the formula on reconstitution time, stability of the drug solution obtained by reconstitution, and storage stability of formulation in the case of the same amount of human serum albumin specified in the formula. The reconstitution results indicate that, the reconstitution time of the formulation significantly decreased with the increase of the amount of lactose specified in the formula, and the drug solution obtained by reconstitution was able to remain stable for no less than 8 h. As can be seen from the results of stability examination, the growth rate of the related substances significantly slowed with the increase of the amount of lactose specified in the formula, and the storage stability of the formulation significantly increased with the increase of the amount of lactose specified in the formula. Therefore, lactose can shorten the reconstitution time of the formulation and increase the stability of the formulation.

### Example 8

### 8.1 Formula Information

| Drug Substance and | | | | | | | | |
|---|---|---|---|---|---|---|---|---|
| Excipients | Formula 8-1 | | Formula 8-2 | | Formula 8-3 | | Formula 8-4 | |
| Fosaprepitant dimeglumine | 1.545 | g | 1.545 | g | 1.545 | g | 1.545 | g |
| Palonosetron hydrochloride | 1.764 | mg | 1.764 | mg | 1.764 | mg | 1.764 | mg |
| Human serum albumin | 600 | mg | 600 | mg | 600 | mg | 600 | mg |
| Lactose | 0 | mg | 1200 | mg | 1800 | mg | 2400 | mg |

| | | | | |
|---|---|---|---|---|
| 0.5 mol/L sodium hydroxide | q.s. | q.s. | q.s. | q.s. |
| Purified water | To 36 mL | To 36 mL | To 36 mL | To 36 mL |

### 8.2 Preparation Process

Lactose at an amount specified in the formula was weighed out and added to a 100 mL beaker (containing a stir bar), water (20-60° C.) that was 80% of the formulating amount was added, and then the stirring was started to dissolve lactose.

The drug solution was cooled to 10-30° C., and palonosetron hydrochloride and human serum albumin each at an amount specified in the formula were weighed out and added to the above 100 ml beaker.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. The vials were partially stoppered, transferred to a lyophilizer, and lyophilized for 30 h to obtain finished products.

### 8.3 Reconstitution Process

One vial in each of the above 4 batches of lyophilized samples and a proper amount of 0.9% sodium chloride injection were left to stand to reach room temperature.

5 mL of 0.9% sodium chloride injection was drawn with a syringe (5 mL) and slowly injected into each of the 4 vials of samples along the wall to reconstitute the samples.

The reconstituted samples were each drawn with a syringe (5 mL) and injected into an infusion bottle containing 145 mL of 0.9% sodium chloride injection to be diluted to 150 mL, the infusion bottle was slightly turned 3 times to uniformly mix the drug solution, and the drug solution was left to stand at room temperature and observed whether visible particles were precipitated from the drug solution.

### 8.4 Results of Reconstitution Experiment

| | | Amount of | | |
|---|---|---|---|---|
| | Amount of | human serum | | |
| | Lactose | albumin | | |
| Sample (Formula | specified in | specified in the | Reconstitution | Observation of Visible |
| No.) | the formula | formula | Time (min) | Particles |
| Formula 8-1 | 0 mg/vial | 100 mg/vial | 13 | Being clear and free of |
| | | | | visible particles after |
| | | | | being left to stand at |
| | | | | room temperature for |
| | | | | 8 h |
| Formula 8-2 | 200 mg/vial | 100 mg/vial | 5 | Being clear and free of |
| | | | | visible particles after |
| | | | | being left to stand at |
| | | | | room temperature for |
| | | | | 8 h |
| Formula 8-3 | 300 mg/vial | 100 mg/vial | 3.5 | Being clear and free of |
| | | | | visible particles after |
| | | | | being left to stand at |
| | | | | room temperature for |
| | | | | 8 h |
| Formula 8-4 | 400 mg/vial | 100 mg/vial | 2 | Being clear and free of |
| | | | | visible particles after |
| | | | | being left to stand at |
| | | | | room temperature for |
| | | | | 8 h |

### 8.5 Experiment on Storage Stability Examination and Results

The above 4 batches of lyophilized samples were subjected to stability examination at 40° C. and 25° C. and compared, and the results are as follows:

Results of destruction experiment at 40° C.

| Related | | | | | |
|---|---|---|---|---|---|
| Substance | Examination | Sample (Formula No.) | | | |
| % | Conditions | Formula 8-1 | Formula 8-2 | Formula 8-3 | Formula 8-4 |
| Impurity C | 0 days | 0.130 | 0.111 | 0.107 | 0.113 |
| (aprepitant) | 40° C., 6 days | 0.900 | 0.561 | 0.523 | 0.502 |
| | 40° C., 15 days | 1.499 | 0.806 | 0.794 | 0.856 |
| Total | 0 days | 0.275 | 0.238 | 0.231 | 0.241 |
| impurity | 40° C., 6 days | 1.107 | 0.677 | 0.667 | 0.612 |
| | 40° C., 15 days | 1.775 | 0.941 | 0.923 | 0.962 |

Results of accelerated experiment at 25° C.

| | | Sample (Formula No.) | | | |
|---|---|---|---|---|---|
| Related | Examination | Formula | | | |
| Substance % | Conditions | 8-1 | Formula 8-2 | Formula 8-3 | Formula 8-4 |
| Impurity C | 0 days | 0.130 | 0.111 | 0.107 | 0.113 |
| (aprepitant) | 25° C., 1 | 0.482 | 0.331 | 0.371 | 0.337 |
| | month | | | | |
| | 25° C., 2 | 0.701 | 0.461 | 0.427 | 0.447 |
| | months | | | | |
| | 25° C., 3 | 0.934 | 0.606 | 0.567 | 0.581 |
| | months | | | | |
| | 25° C., 6 | 1.250 | 0.770 | 0.700 | 0.7000 |
| | months | | | | |
| Total | 0 days | 0.275 | 0.238 | 0.231 | 0.241 |
| impurity | 25° C., 1 | 0.621 | 0.459 | 0.423 | 0.414 |
| | month | | | | |
| | 25° C., 2 | 0.841 | 0.533 | 0.507 | 0.498 |
| | months | | | | |
| | 25° C., 3 | 1.120 | 0.780 | 0.660 | 0.660 |
| | months | | | | |
| | 25° C., 6 | 1.485 | 0.895 | 0.814 | 0.794 |
| | months | | | | |

### 8.6 Conclusion

This example further compares, for a compound formulation comprising palonosetron hydrochloride, the influences of different amounts of lactose in the formula on reconstitution time, stability of the drug solution obtained by reconstitution, and storage stability of formulation in the case of the same amount of human serum albumin specified in the formula. As can be seen from the reconstitution results, similar to the rule of the single-drug formulation in Example 5, the reconstitution time of the formulation of this example significantly decreased with the increase of the amount of lactose specified in the formula, and the drug solution obtained by reconstitution was able to remain stable for no less than 8 h.

It can be observed from stability examination that when the amount of human serum albumin specified in the formula of the 4 batches of samples remained the same, the growth rate of the related substances in the formulation decreased with the increase of the amount of lactose specified in the formula, and the storage stability of the formulation increased with the increase of the amount of lactose specified in the formula.

### Example 9

### 9.1 Formula Information

| Drug Substance | Formula | | Formula | | Formula | | Formula | | Formula | | Formula | |
|---|---|---|---|---|---|---|---|---|---|---|---|---|
| and Excipients | 9-1 | | 9-2 | | 9-3 | | 9-4 | | 9-5 | | 9-6 | |
| Fosaprepitant | 1.288 | g | 1.288 | g | 1.288 | g | 1.288 | g | 1.288 | g | 1.288 | g |
| dimeglumine | | | | | | | | | | | | |
| Human serum | 200 | mg | 300 | mg | 500 | mg | 1000 | mg | 1500 | mg | 2000 | mg |
| albumin | | | | | | | | | | | | |
| Lactose | 2000 | mg | 2000 | mg | 2000 | mg | 2000 | mg | 2000 | mg | 2000 | mg |
| 0.5 mol/L | q.s. | | q.s. | | q.s. | | q.s. | | q.s. | | q.s. | |
| sodium | | | | | | | | | | | | |
| hydroxide | | | | | | | | | | | | |

| | | | | | | |
|---|---|---|---|---|---|---|
| Purified water | To 30 mL | To 30 mL | To 30 mL | To 30 mL | To 30 mL | To 30 mL |

### 9.2 Preparation Process

Lactose at an amount specified in the formula was weighed out and added to a 100 mL beaker (containing a stir bar), a proper amount of water (20-60° C.) was added, and then the stirring was started to dissolve lactose.

The drug solution was cooled to 10-30° C., and human serum albumin at an amount specified in the formula was weighed out and added to the above 100 ml beaker. The total amount of the solution in this step was 88.33% of the formulating amount.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 4 vials in each batch. The vials were partially stoppered, transferred to a lyophilizer, and lyophilized for 30 h to obtain finished products.

### 9.3 Reconstitution Process

One vial in each of the above 6 batches of lyophilized samples and a proper amount of 0.9% sodium chloride injection were left to stand to reach room temperature.

5 mL of 0.9% sodium chloride injection was drawn with a syringe (5 mL) and slowly injected into each of the 6 vials of samples along the wall to reconstitute the samples, and the reconstitution time was recorded.

### 9.4 Results of Reconstitution Experiment

| | | Amount of | | Number of | |
|---|---|---|---|---|---|
| | Amount of | human serum | | Bubbles | |
| Sample | lactose | albumin | | Generated | |
| (Formula | specified in | specified in | Reconstitution | During | Influence of |
| No.) | the formula | the formula | Time (min) | Reconstitution | Bubbles |
| Formula 9-1 | 400 mg/vial | 40 mg/vial | 1.5 | + | Does not |
| | | | | | influence |
| | | | | | reconstitution |
| Formula 9-2 | 400 mg/vial | 60 mg/vial | 2 | ++ | Does not |
| | | | | | influence |
| | | | | | reconstitution |
| Formula 9-3 | 400 mg/vial | 100 mg/vial | 3.3 | +++ | Does not |
| | | | | | influence |
| | | | | | reconstitution |
| Formula 9-4 | 400 mg/vial | 200 mg/vial | 11 | ++++ | Has little |
| | | | | | influence on |
| | | | | | reconstitution |
| Formula 9-5 | 400 mg/vial | 300 mg/vial | 24 | +++++ | Has certain |
| | | | | | influence on |
| | | | | | reconstitution |
| Formula 9-6 | 400 mg/vial | 400 mg/vial | 36 | ++++++ | Does not |
| | | | | | dissipate after a |
| | | | | | long time (no |
| | | | | | less than 2 h) |
| | | | | | and has great |
| | | | | | influence on |
| | | | | | reconstitution |

Note:
more “+” sign indicates more bubbles generated during reconstitution.

### 9.5 Conclusion

This example compares the influences of different amounts of human serum albumin in the formula on reconstitution time in the case of the same amount of lactose specified in the formula (400 mg/vial). The results indicate that, in the presence of a lyophilization excipient, the reconstitution time of the formulation also significantly increased with the increase of the amount of human serum albumin specified in the formula, and the number of bubbles generated during the reconstitution process also significantly increased with the increase of the amount of human serum albumin specified in the formula, wherein when the amount of human serum albumin specified in the formula was 400 mg/vial, a large number of bubbles were generated and did not dissipate after a long time (no less than 2 h), which had great influence on reconstitution, with the reconstitution time being more than 30 min.

### Example 10

### 10.1 Formula Information

| Drug Substance | Formula | | Formula | | Formula | | Formula | | Formula | | Formula | |
|---|---|---|---|---|---|---|---|---|---|---|---|---|
| and Excipients | 10-1 | | 10-2 | | 10-3 | | 10-4 | | 10-5 | | 10-6 | |
| Fosaprepitant | 1.288 | g | 1.288 | g | 1.288 | g | 1.288 | g | 1.288 | g | 1.288 | g |
| dimeglumine | | | | | | | | | | | | |
| Palonosetron | 1.470 | mg | 1.470 | mg | 1.470 | mg | 1.470 | mg | 1.470 | mg | 1.470 | mg |
| hydrochloride | | | | | | | | | | | | |
| Human serum | 200 | mg | 300 | mg | 500 | mg | 1000 | mg | 1500 | mg | 2000 | mg |
| albumin | | | | | | | | | | | | |
| Lactose | 1750 | mg | 1750 | mg | 1750 | mg | 1750 | mg | 1750 | mg | 1750 | mg |
| 0.5 mol/L | q.s. | | q.s. | | q.s. | | q.s. | | q.s. | | q.s. | |
| sodium | | | | | | | | | | | | |
| hydroxide | | | | | | | | | | | | |

| | | | | | | |
|---|---|---|---|---|---|---|
| Purified water | To 30 mL | To 30 mL | To 30 mL | To 30 mL | To 30 mL | To 30 mL |

### 10.2 Preparation Process

Lactose at an amount specified in the formula was weighed out and added to a 100 mL beaker (containing a stir bar), a proper amount of water (20-60° C.) was added, and then the stirring was started to dissolve lactose.

The drug solution was cooled to 10-30° C., and palonosetron hydrochloride and human serum albumin each at an amount specified in the formula were weighed out and added to the above 100 ml beaker. The total amount of the solution in this step was 88.33% of the formulating amount.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 4 vials in each batch. The vials were partially stoppered, transferred to a lyophilizer, and lyophilized for 30 h to obtain finished products.

### 10.3 Reconstitution Process

One vial in each of the above 6 batches of lyophilized samples and a proper amount of 0.9% sodium chloride injection were left to stand to reach room temperature.

5 mL of 0.9% sodium chloride injection was drawn with a syringe (5 mL) and slowly injected into each of the 6 vials of samples along the wall to reconstitute the samples, and the reconstitution time was recorded.

### 10.4 Results of Reconstitution Experiment

| | | Amount of | | | |
|---|---|---|---|---|---|
| | Amount of | human serum | | Number of | |
| | Lactose | albumin | | Bubbles | |
| Sample | specified | specified | | Generated | |
| (Formula | in the | in the | Reconstitution | During | Influence of |
| No.) | formula | formula | Time (min) | Reconstitution | Bubbles |
| Formula 10-1 | 350 mg/vial | 40 mg/vial | 1.5 | + | Does not |
| | | | | | influence |
| | | | | | reconstitution |
| Formula 10-2 | 350 mg/vial | 60 mg/vial | 1.8 | ++ | Does not |
| | | | | | influence |
| | | | | | reconstitution |
| Formula 10-3 | 350 mg/vial | 100 mg/vial | 3.3 | +++ | Does not |
| | | | | | influence |
| | | | | | reconstitution |
| Formula 10-4 | 350 mg/vial | 200 mg/vial | 8 | ++++ | Has little |
| | | | | | influence on |
| | | | | | reconstitution |
| Formula 10-5 | 350 mg/vial | 300 mg/vial | 24 | +++++ | Has certain |
| | | | | | influence on |
| | | | | | reconstitution |
| Formula 10-6 | 350 mg/vial | 400 mg/vial | 33 | ++++++ | Does not |
| | | | | | dissipate after a |
| | | | | | long time (no |
| | | | | | less than 2 h) |
| | | | | | and has great |
| | | | | | influence on |
| | | | | | reconstitution |

Note:
more “+” sign indicates more bubbles generated during reconstitution.

### 10.5 Conclusion

This example further compares, for a compound formulation comprising palonosetron hydrochloride, the influences of different amounts of human serum albumin in the formula on reconstitution time in the case of the same amount of lactose specified in the formula (350 mg/vial). The results indicate that, similar to the rule of the single-drug formulation in Example 9, in the presence of a lyophilization excipient, the reconstitution time of the compound formulation of this example significantly increased with the increase of the amount of human serum albumin specified in the formula, and the number of bubbles generated during the reconstitution process also significantly increased with the increase of the amount of human serum albumin specified in the formula, wherein when the amount of human serum albumin specified in the formula was 400 mg/vial, a large number of bubbles were generated and did not dissipate after a long time (no less than 2 h), which had great influence on reconstitution, with the reconstitution time being more than 30 min.

### Example 11

### 11.1 Formula Information

| Drug Substance | | | | | | | | |
|---|---|---|---|---|---|---|---|---|
| and Excipients | Formula 11-1 | | Formula 11-2 | | Formula 11-3 | | Formula 11-4 | |
| Fosaprepitant dimeglumine | 1.545 | g | 1.545 | g | 1.545 | g | 1.545 | g |
| Palonosetron hydrochloride | 1.764 | mg | 1.764 | mg | 1.764 | mg | 1.764 | mg |
| Human serum albumin | 600 | mg | 600 | mg | 600 | mg | 600 | mg |
| Mannitol | 0 | mg | 600 | mg | 1200 | mg | 1800 | mg |
| 0.5 mol/L sodium hydroxide | q.s. | | q.s. | | q.s. | | q.s. | |
| Purified water | To 36 | mL | To 36 | mL | To 36 | mL | To 36 | mL |

### 11.2 Preparation Process

Mannitol, palonosetron hydrochloride, and human serum albumin each at an amount specified in the formula were weighed out and added to a 100 mL beaker (containing a stir bar), water (10-30° C.) that was 80% of the formulating amount was added, and then the stirring was started to dissolve the drug substance and excipients.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. The vials were partially stoppered, transferred to a lyophilizer, and lyophilized for 30 h to obtain finished products.

### 11.3 Reconstitution Process

One vial in each of the above 4 batches of lyophilized samples and a proper amount of 0.9% sodium chloride injection were left to stand to reach room temperature.

5 mL of 0.9% sodium chloride injection was drawn with a syringe (5 mL) and slowly injected into each of the 4 vials of samples along the wall to reconstitute the samples, and the reconstitution time was recorded.

The reconstituted samples were each drawn with a syringe (5 mL) and injected into an infusion bottle containing 145 mL of 0.9% sodium chloride injection to be diluted to 150 mL, the infusion bottle was slightly turned 3 times to uniformly mix the drug solution, and the drug solution was left to stand at room temperature and observed whether visible particles were precipitated from the drug solution.

### 11.4 Results of Reconstitution Experiment

| | | Amount of | | |
|---|---|---|---|---|
| | Amount of | human serum | | |
| Sample | mannitol | albumin | | Observation |
| (Formula | specified in | specified in | Reconstitution | of Visible |
| No.) | the formula | the formula | Time (min) | Particles |
| Formula 11-1 | 0 mg/vial | 100 mg/vial | 12 | Being clear and free of |
| | | | | visible particles after |
| | | | | being left to stand at room |
| | | | | temperature for 8 h |
| Formula 11-2 | 100 mg/vial | 100 mg/vial | 4 | Being clear and free of |
| | | | | visible particles after |
| | | | | being left to stand at room |
| | | | | temperature for 8 h |
| Formula 11-3 | 200 mg/vial | 100 mg/vial | 3 | Being clear and free of |
| | | | | visible particles after |
| | | | | being left to stand at room |
| | | | | temperature for 8 h |
| Formula 11-4 | 300 mg/vial | 100 mg/vial | 2.5 | Being clear and free of |
| | | | | visible particles after |
| | | | | being left to stand at room |
| | | | | temperature for 8 h |

### 11.5 Experiment on Storage Stability Examination and Results

The above 4 batches of lyophilized samples were subjected to stability examination at 40° C. and 25° C. and compared, and the results are as follows:

Results of destruction experiment at 40° C.

| Related | | Sample (Formula No.) | | | |
|---|---|---|---|---|---|
| Substance | Examination | Formula | Formula | Formula | Formula |
| % | Conditions | 11-1 | 11-2 | 11-3 | 11-4 |
| Impurity C | 0 days | 0.211 | 0.184 | 0.186 | 0.183 |
| (aprepitant) | 40° C., 9 days | 1.118 | 0.838 | 0.982 | 0.994 |
| | 40° C., 15 days | 1.570 | 1.308 | 1.448 | 1.430 |
| Total | 0 days | 0.322 | 0.294 | 0.292 | 0.289 |
| impurity | 40° C., 9 days | 1.367 | 0.989 | 1.172 | 1.192 |
| | 40° C., 15 days | 1.822 | 1.395 | 1.634 | 1.603 |

Results of accelerated experiment at 25° C.

| Related | | Sample (Formula No.) | | | |
|---|---|---|---|---|---|
| Substance | Examination | Formula | Formula | Formula | Formula |
| % | Conditions | 11-1 | 11-2 | 11-3 | 11-4 |
| Impurity C | 0 days | 0.211 | 0.184 | 0.186 | 0.183 |
| (aprepitant) | 25° C., 1 month | 0.544 | 0.340 | 0.418 | 0.450 |
| | 25° C., 2 months | 0.750 | 0.455 | 0.564 | 0.604 |
| | 25° C., 3 months | 0.980 | 0.590 | 0.776 | 0.810 |
| Total | 0 days | 0.322 | 0.294 | 0.292 | 0.289 |
| impurity | 25° C., 1 month | 0.686 | 0.434 | 0.517 | 0.558 |
| | 25° C., 2 months | 0.971 | 0.594 | 0.715 | 0.748 |
| | 25° C., 3 months | 1.190 | 0.690 | 0.930 | 0.960 |

### 11.6 Conclusion

This example compares the influences of different amounts of mannitol in the formula on reconstitution time, stability of the drug solution obtained by reconstitution, and storage stability of formulation in the case of the same amount of human serum albumin specified in the formula (100 mg/vial). As can be seen from the reconstitution results, the reconstitution time of the formulation significantly decreased with the increase of the amount of mannitol specified in the formula, and the drug solution obtained by reconstitution was able to remain stable for no less than 8 h. Comparing this example further with Example 5, it can be seen that the same amount of mannitol exhibits a better effect on reducing reconstitution time than lactose. As can be seen from the results of stability examination, the addition of mannitol in the formula can increase the storage stability of the sample to a certain extent.

### Example 12

### 12.1 Formula Information

| Drug Substance | Formula | | Formula | | Formula | | Formula | | Formula | | Formula | |
|---|---|---|---|---|---|---|---|---|---|---|---|---|
| and Excipients | 12-1 | | 12-2 | | 12-3 | | 12-4 | | 12-5 | | 12-6 | |
| Fosaprepitant | 1.030 | g | 1.030 | g | 1.030 | g | 1.030 | g | 1.030 | g | 1.030 | g |
| dimeglumine | | | | | | | | | | | | |
| Human serum | 160 | mg | 240 | mg | 400 | mg | 800 | mg | 1200 | mg | 1600 | mg |
| albumin | | | | | | | | | | | | |
| Mannitol | 400 | mg | 400 | mg | 400 | mg | 400 | mg | 400 | mg | 400 | mg |
| 0.5 mol/L | q.s. | | q.s. | | q.s. | | q.s. | | q.s. | | q.s. | |
| sodium | | | | | | | | | | | | |
| hydroxide | | | | | | | | | | | | |

| | | | | | | |
|---|---|---|---|---|---|---|
| Purified water | To 24 mL | To 24 mL | To 24 mL | To 24 mL | To 24 mL | To 24 mL |

### 12.2 Formulating Process

Mannitol at an amount specified in the formula was weighed out and added to a 100 mL beaker (containing a stir bar), a proper amount of water (10-30° C.) and human serum albumin at an amount specified in the formula were added, and then the stirring was started to dissolve mannitol. The total amount of the solution in this step was 88.33% of the formulating amount.

Fosaprepitant dimeglumine at an amount specified in the formula was weighed out and added to the above 100 ml beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 3 vials in each batch. The vials were partially stoppered, transferred to a lyophilizer, and lyophilized for 26.5 h to obtain finished products.

### 12.3 Reconstitution Process

One vial in each of the above 6 batches of lyophilized samples and a proper amount of 0.9% sodium chloride injection were left to stand to reach room temperature.

5 mL of 0.9% sodium chloride injection was drawn with a syringe (5 mL) and slowly injected into each of the 6 vials of samples along the wall to reconstitute the samples, and the reconstitution time was recorded.

### 12.4 Results of Reconstitution Experiment

| | | Amount of | | Number of | |
|---|---|---|---|---|---|
| | Amount of | human serum | | Bubbles | |
| Sample | mannitol | albumin | | Generated | |
| (Formula | specified in | specified in | Reconstitution | During | Influence of |
| No.) | the formula | the formula | Time (min) | Reconstitution | Bubbles |
| Formula 12-1 | 100 mg/vial | 40 mg/vial | 2 | + | Does not |
| | | | | | influence |
| | | | | | reconstitution |
| Formula 12-2 | 100 mg/vial | 60 mg/vial | 5 | ++ | Does not |
| | | | | | influence |
| | | | | | reconstitution |
| Formula 12-3 | 100 mg/vial | 100 mg/vial | 8 | +++ | Does not |
| | | | | | influence |
| | | | | | reconstitution |
| Formula 12-4 | 100 mg/vial | 200 mg/vial | 12 | ++++ | Has little |
| | | | | | influence on |
| | | | | | reconstitution |
| Formula 12-5 | 100 mg/vial | 300 mg/vial | 22 | +++++ | Has certain |
| | | | | | influence on |
| | | | | | reconstitution |
| Formula 12-6 | 100 mg/vial | 400 mg/vial | 30 | ++++++ | Does not |
| | | | | | dissipate after a |
| | | | | | long time (no |
| | | | | | less than 2 h) |
| | | | | | and has great |
| | | | | | influence on |
| | | | | | reconstitution |

Note:
more “+” sign indicates more bubbles generated during reconstitution.

### 12.5 Conclusion

This example compares the influences of different amounts of human serum albumin in the formula on reconstitution time in the case of the same amount of mannitol specified in the formula (100 mg/vial). As can be seen from the results, the reconstitution time of the formulation of this example significantly increased with the increase of the amount of human serum albumin specified in the formula, and the number of bubbles generated during the reconstitution process also significantly increased with the increase of the amount of human serum albumin specified in the formula, wherein when the amount of human serum albumin specified in the formula was 400 mg/vial, a large number of bubbles were generated and did not dissipate after a long time (no less than 2 h), which had great influence on reconstitution.

### Example 13

### 13.1 Formula Information

| Drug Substance | Formula | | Formula | | Formula | | Formula | | Formula | | Formula | |
|---|---|---|---|---|---|---|---|---|---|---|---|---|
| and Excipients | 13-1 | | 13-2 | | 13-3 | | 13-4 | | 13-5 | | 13-6 | |
| Fosaprepitant | 1.030 | g | 1.030 | g | 1.030 | g | 1.030 | g | 1.030 | g | 1.030 | g |
| dimeglumine | | | | | | | | | | | | |
| Palonosetron | 1.176 | mg | 1.176 | mg | 1.176 | mg | 1.176 | mg | 1.176 | mg | 1.176 | mg |
| hydrochloride | | | | | | | | | | | | |
| Human serum | 160 | mg | 240 | mg | 400 | mg | 800 | mg | 1200 | mg | 1600 | mg |
| albumin | | | | | | | | | | | | |
| Mannitol | 400 | mg | 400 | mg | 400 | mg | 400 | mg | 400 | mg | 400 | mg |
| 0.5 mol/L | q.s. | | q.s. | | q.s. | | q.s. | | q.s. | | q.s. | |
| sodium | | | | | | | | | | | | |
| hydroxide | | | | | | | | | | | | |

| | | | | | | |
|---|---|---|---|---|---|---|
| Purified water | To 24 mL | To 24 mL | To 24 mL | To 24 mL | To 24 mL | To 24 mL |

### 13.2 Formulating Process

Mannitol and palonosetron hydrochloride each at an amount specified in the formula were weighed out and added to a 100 mL beaker (containing a stir bar), a proper amount of water (10-30° C.) and human serum albumin at an amount specified in the formula were added, and then the stirring was started to dissolve the drug substance and excipients. The total amount of the solution in this step was 88.33% of the formulating amount.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. Each formula was 1 batch, and there were 3 vials in each batch. The vials were partially stoppered, transferred to a lyophilizer, and lyophilized for 26.5 h to obtain finished products.

### 13.3 Reconstitution Process

One vial in each of the above 6 batches of lyophilized samples and a proper amount of 0.9% sodium chloride injection were left to stand to reach room temperature.

5 mL of 0.9% sodium chloride injection was drawn with a syringe (5 mL) and slowly injected into each of the 6 vials of samples along the wall to reconstitute the samples, and the reconstitution time was recorded.

### 13.4 Results of Reconstitution Experiment

| | Amount of | Amount of | | Number of | |
|---|---|---|---|---|---|
| | Mannitol | human serum | | Bubbles | |
| Sample | specified | albumin | | Generated | |
| (Formula | in the | specified in | Reconstitution | During | Influence of |
| No.) | formula | the formula | Time (min) | Reconstitution | Bubbles |
| Formula 13-1 | 100 mg/vial | 40 mg/vial | 2.5 | + | Does not |
| | | | | | influence |
| | | | | | reconstitution |
| Formula 13-2 | 100 mg/vial | 60 mg/vial | 4.5 | ++ | Does not |
| | | | | | influence |
| | | | | | reconstitution |
| Formula 13-3 | 100 mg/vial | 100 mg/vial | 6 | +++ | Does not |
| | | | | | influence |
| | | | | | reconstitution |
| Formula 13-4 | 100 mg/vial | 200 mg/vial | 9 | ++++ | Has little |
| | | | | | influence on |
| | | | | | reconstitution |
| Formula 13-5 | 100 mg/vial | 300 mg/vial | 21 | +++++ | Has certain |
| | | | | | influence on |
| | | | | | reconstitution |
| Formula 13-6 | 100 mg/vial | 400 mg/vial | 29 | ++++++ | Does not |
| | | | | | dissipate after a |
| | | | | | long time (no |
| | | | | | less than 2 h) |
| | | | | | and has great |
| | | | | | influence on |
| | | | | | reconstitution |

Note:
more “+” sign indicates more bubbles generated during reconstitution.

### 13.5 Conclusion

This example further compares, for a compound formulation comprising palonosetron hydrochloride, the influences of different amounts of human serum albumin in the formula on reconstitution time in the case of the same amount of mannitol specified in the formula (100 mg/vial). As can be seen from the results, similar to the rule of the single-drug formulation in Example 10, the reconstitution time of the compound formulation of this example significantly increased with the increase of the amount of human serum albumin specified in the formula, and the number of bubbles generated during the reconstitution process also significantly increased with the increase of the amount of human serum albumin specified in the formula, wherein when the amount of human serum albumin specified in the formula was 400 mg/vial, a large number of bubbles were generated and did not dissipate after a long time (no less than 2 h), which had great influence on reconstitution.

### Example 14

### 14.1 Formula Information

| Drug Substance | | | | | | |
|---|---|---|---|---|---|---|
| and Excipients | Formula 14-1 | | Formula 14-2 | | Formula 14-3 | |
| Fosaprepitant | 1.545 | g | 1.545 | g | 1.545 | g |
| dimeglumine | | | | | | |
| Human serum albumin | 600 | mg | 600 | mg | 600 | mg |
| Lactose | 1800 | mg | 1800 | mg | 1800 | mg |
| 0.5 mol/L sodium | An amount enabling | | An amount enabling | | An amount enabling | |
| hydroxide | adjustment of pH to 8.0 | | adjustment of pH to 8.5 | | adjustment of pH to 9.0 | |
| Purified water | To 36 | mL | To 36 | mL | To 36 | mL |

### 14.2 Preparation Process

Lactose at an amount specified in the formula was weighed out and added to a 100 ml beaker (containing a stir bar), water (20-60° C.) that was 80% of the formulating amount was added, and then the stirring was started to dissolve lactose.

The drug solution was cooled to 10-30° C., and human serum albumin at an amount specified in the formula was weighed out and added to the above 100 ml beaker.

Fosaprepitant dimeglumine at an amount specified in the formula was weighed out and added to the above 100 ml beaker to be dissolved.

A 0.5 mol/L sodium hydroxide solution was added dropwise slowly; for the 3 batches of samples, the pH values of drug solution were adjusted to 8.0, 8.5, and 9.0, respectively.

The volume of the drug solution was brought to the total formulating amount by supplementing with water, and then the drug solution was uniformly stirred, filtered through a 0.22 μm PES filter, and filled in 20 mL vials at an amount of 6 mL per vial. The vials were partially stoppered, transferred to a lyophilizer, and lyophilized for 30 h to obtain finished products.

### 14.3 Experiment on Storage Stability Examination and Results

The above 3 batches of lyophilized samples were subjected to stability examination at 40° C. and 25° C. and compared, and the results are as follows:

Results of destruction experiment at 40° C.

| | |
|---|---|
| Related | Sample (Formula No.) |

| Substance | Examination | Formula | Formula | Formula |
|---|---|---|---|---|
| % | Conditions | 14-1 | 14-2 | 14-3 |
| Impurity C | 0 days | 0.103 | 0.094 | 0.088 |
| (aprepitant) | 40° C., 6 days | 0.569 | 0.484 | 0.455 |
| | 40° C., 10 days | 0.705 | 0.607 | 0.573 |
| | 40° C., 15 days | 0.872 | 0.745 | 0.705 |
| Total | 0 days | 0.160 | 0.149 | 0.145 |
| impurity | 40° C., 6 days | 0.646 | 0.565 | 0.530 |
| | 40° C., 10 days | 0.807 | 0.702 | 0.672 |
| | 40° C., 15 days | 1.037 | 0.905 | 0.852 |

Results of accelerated experiment at 25° C.

| Related | | Sample (Formula No.) | | |
|---|---|---|---|---|
| Substance | Examination | Formula | Formula | Formula |
| % | Conditions | 14-1 | 14-2 | 14-3 |
| Impurity C | 0 days | 0.103 | 0.094 | 0.088 |
| (aprepitant) | 25° C., 1 month | 0.362 | 0.321 | 0.296 |
| | 25° C., 2 months | 0.490 | 0.431 | 0.396 |
| | 25° C., 3 months | 0.520 | 0.463 | 0.428 |
| | 25° C., 6 months | 0.784 | 0.688 | 0.632 |
| Total | 0 days | 0.160 | 0.149 | 0.145 |
| impurity | 25° C., 1 month | 0.483 | 0.447 | 0.411 |
| | 25° C., 2 months | 0.564 | 0.503 | 0.476 |
| | 25° C., 3 months | 0.625 | 0.561 | 0.522 |
| | 25° C., 6 months | 0.797 | 0.710 | 0.648 |

### 14.4 Conclusion

This example examines the influence of pH on stability and compares the results of storage stability of formulation when adjusted to different pH values. As can be seen from the results of stability examination, the storage stability of the formulation when adjusted to different pH values was ranked as pH 9.0>pH 8.5>pH 8.0. Therefore, the pH value is preferably adjusted to be in the range of 8.5-9.0.

### Example 15

### 15.1 Formula Information

| Drug Substance | | |
|---|---|---|
| and Excipients | Formula 15-1 | |
| Fosaprepitant dimeglumine | 1.545 | g |
| Human serum albumin | 840 | mg |
| Lactose | 1800 | mg |
| 0.5 mol/L sodium hydroxide | q.s. | |
| Purified water | To 36 | mL |

### 15.2 Preparation Process

Lactose at an amount specified in the formula was weighed out and added to a 100 ml beaker (containing a stir bar), water (20-60° C.) that was 80% of the formulating amount was added, and then the stirring was started to dissolve lactose.

The drug solution was cooled to 10-30° C., and human serum albumin at an amount specified in the formula was weighed out and added to the above 100 ml beaker.

Fosaprepitant dimeglumine at an amount specified in the formula was weighed out and added to the above 100 mL beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. The vials were partially stoppered, transferred to a lyophilizer, and lyophilized for 30 h to obtain finished products.

### 15.3 Reconstitution Process and Results

One vial of the above lyophilized sample and a proper amount of 0.9% sodium chloride injection were left to stand to reach room temperature.

5 mL of 0.9% sodium chloride injection was drawn with a syringe (5 mL) and slowly injected into the sample along the wall to reconstitute the sample, and the reconstitution time was about 6 min.

The reconstituted sample was drawn with a syringe (5 mL) and injected into an infusion bottle containing 145 mL of 0.9% sodium chloride injection to be diluted to 150 mL, the infusion bottle was slightly turned 3 times to uniformly mix the drug solution, and the drug solution was left to stand at room temperature and observed whether visible particles were precipitated from the drug solution. Result: the drug solution remained clear and no visible particles were observed for 24 h.

### 15.4 Experiment on Storage Stability Examination and Results

The above lyophilized sample was subjected to stability examination at 25° C. for 12 months, and the results are as follows:

Results of experiment on stability examination at 25° C.

| Sample | Examination | Major Related Substance |
|---|---|---|
| (Formula No.) | Conditions | Impurity C (aprepitant)% |
| Formula 15-1 | 0 days | 0.081 |
| | 25° C., 1 month | 0.320 |
| | 25° C., 2 months | 0.426 |
| | 25° C., 3 months | 0.520 |
| | 25° C., 6 months | 0.752 |
| | 25° C., 9 months | 0.950 |
| | 25° C., 12 months | 1.125 |

Conclusion: the mass percentage of the impurity C (aprepitant) of the formulation of this example was still no more than 2% after being at 25° C. for 12 months, and the long-term storage stability of the formulation is high.

### Example 16

### 16.1 Formula Information

| Drug Substance | Formula 16-1 | |
|---|---|---|
| and Excipients | (80 vials) | |
| Fosaprepitant dimeglumine | 20.605 | g |
| Palonosetron hydrochloride | 23.520 | mg |
| Human serum albumin | 11.200 | g |
| Lactose | 32.000 | g |
| 0.5 mol/L sodium hydroxide | q.s. | |
| Purified water | To 480 | mL |

### 16.2 Preparation Process

Lactose at an amount specified in the formula was weighed out and added to a 1 L beaker (containing a stir bar), water (20-60° C.) that was 80% of the formulating amount was added, and then the stirring was started to dissolve lactose.

The drug solution was cooled to 10-30° C., and human serum albumin and palonosetron hydrochloride each at an amount specified in the formula were weighed out and added to the above 1 L beaker.

Fosaprepitant dimeglumine at an amount specified in the formula was weighed out and added to the above 1 L beaker to be dissolved.

A 0.5 mol/L sodium hydroxide solution was added dropwise slowly to adjust the pH of the drug solution to 8.50-9.00.

The volume of the drug solution was brought to the total formulating amount by supplementing with water, and then the drug solution was uniformly stirred, filtered through a 0.22 μm PES filter, and filled in 20 mL vials at an amount of 6 mL per vial. The vials were partially stoppered, transferred to a lyophilizer, and lyophilized for 30 h to obtain finished products.

### 16.3 Reconstitution Process and Results

Two vials of the above lyophilized sample and a proper amount of 0.9% sodium chloride injection were left to stand to reach room temperature.

5 mL of 0.9% sodium chloride injection was drawn with a syringe (5 mL) and slowly injected into the samples along the wall to reconstitute the samples, and the reconstitution time was about 7.5 min and about 8 min, respectively.

One vial of drug solution obtained by reconstitution was measured for pH, and the result was 8.75.

Another vial of reconstituted sample was drawn with a syringe (5 mL) and injected into an infusion bottle containing 145 mL of 0.9% sodium chloride injection to be diluted to 150 mL, the infusion bottle was slightly turned 3 times to uniformly mix the drug solution, and the drug solution was left to stand at room temperature and observed whether visible particles were precipitated from the drug solution. Result: the drug solution remained clear and no visible particles were observed for 24 h.

### 16.4 Experiment on Storage Stability Examination and Results

The above lyophilized sample was subjected to stability examination at 25° C., and the results are as follows:

Results of experiment on stability examination at 25° C.

| Sample | Examination | Major Related Substance |
|---|---|---|
| (Formula No.) | Conditions | Impurity C (aprepitant)% |
| Formula 16-1 | 0 days | 0.100 |
| | 25° C., 1 month | 0.280 |
| | 25° C., 2 months | 0.405 |
| | 25° C., 3 months | 0.500 |
| | 25° C., 6 months | 0.750 |

Conclusion: in this example, the scale-up preparation was carried out, and the resulting formulation still had relatively short reconstitution time, good stability of the drug solution obtained by reconstitution, and good storage stability. Scaling up the preparation had a relatively small influence on the reconstitution time, stability of the drug solution obtained by reconstitution, and storage stability, and the preparation method of the present invention is suitable for large-scale production.

Finally, it should be emphasized that the above examples are only preferred examples of the present invention and are not intended to limit the present invention. Any modification, equivalent replacement, improvement, and the like made without departing from the spirit and principle of the present invention shall fall within the protection scope of the present invention.

### INDUSTRIAL APPLICABILITY

The lyophilized formulations provided by the present invention feature short reconstitution time, good storage stability, significant therapeutic effects, and few toxic and side effects and can be used for treating and preventing tumor chemotherapy-induced nausea and vomiting. The preparation method and the use method of the lyophilized formulations provided by the present invention are simple.

1. A lyophilized formulation, comprising fosaprepitant or a pharmaceutically acceptable salt thereof and human serum albumin, wherein the weight ratio of fosaprepitant or the pharmaceutically acceptable salt thereof to the human serum albumin in the lyophilized formulation is 1:0.2 to 1:2; preferably, the weight ratio of fosaprepitant or the pharmaceutically acceptable salt thereof to the human serum albumin in the lyophilized formulation is 1:0.3 to 1:1.3.

2. The lyophilized formulation according to claim 1, further comprising palonosetron or a pharmaceutically acceptable salt thereof.

3. The lyophilized formulation according to claim 1, further comprising a lyophilization excipient, wherein the weight ratio of fosaprepitant or the pharmaceutically acceptable salt thereof to the lyophilization excipient in the lyophilized formulation is 1:0.1 to 1:5; preferably, the weight ratio of fosaprepitant or the pharmaceutically acceptable salt thereof to the lyophilization excipient in the lyophilized formulation is 1:0.4 to 1:3;
the lyophilization excipient is selected from one or more of mannitol, sorbitol, inositol, dextran, maltodextrin, ß-cyclodextrin, polyethylene glycol-4000, polyoxyethylene pyrrolidone, sucrose, maltose, lactose, glucose, trehalose, sodium chloride, potassium chloride, calcium chloride, sodium dihydrogen phosphate, hydrolyzed gelatin, glycine, histidine, lysine, alanine, proline, arginine, aspartic acid, asparagine, or sodium glutamate, and is preferably one or two of mannitol and lactose.

4. The lyophilized formulation according to claim 1, comprising fosaprepitant dimeglumine or a pharmaceutically acceptable salt thereof and the human serum albumin, wherein: each vial of the lyophilized formulation contains 220-270 mg of fosaprepitant dimeglumine and 30-300 mg of the human serum albumin; preferably, each vial of the lyophilized formulation contains 220-270 mg of fosaprepitant dimeglumine and 60-200 mg of the human serum albumin; further preferably, each vial of the lyophilized formulation contains 230-260 mg of fosaprepitant dimeglumine and 100-140 mg of the human serum albumin.

5. The lyophilized formulation according to claim 3, comprising fosaprepitant dimeglumine or a pharmaceutically acceptable salt thereof, the human serum albumin, and the lyophilization excipient, wherein: each vial of the lyophilized formulation contains 220-270 mg of fosaprepitant dimeglumine, 30-300 mg of the human serum albumin, and 10-600 mg of the lyophilization excipient; preferably, each vial of the lyophilized formulation contains 220-270 mg of fosaprepitant dimeglumine, 60-200 mg of the human serum albumin, and 50-500 mg of the lyophilization excipient; further preferably, each vial of the lyophilized formulation contains 230-260 mg of fosaprepitant dimeglumine, 100-140 mg of the human serum albumin, and 100-400 mg of the lyophilization excipient.

6. The lyophilized formulation according to claim 2, comprising fosaprepitant or the pharmaceutically acceptable salt thereof, palonosetron or the pharmaceutically acceptable salt thereof, and the human serum albumin, wherein: each vial of the lyophilized formulation contains 220-270 mg of fosaprepitant dimeglumine, 0.25-0.31 mg of palonosetron hydrochloride, and 30-300 mg of the human serum albumin; preferably, each vial of the lyophilized formulation contains 220-270 mg of fosaprepitant dimeglumine, 0.25-0.31 mg of palonosetron hydrochloride, and 60-200 mg of the human serum albumin; further preferably, each vial of the lyophilized formulation contains 230-260 mg of fosaprepitant dimeglumine, 0.26-0.30 mg of palonosetron hydrochloride, and 100-140.0 mg of the human serum albumin.

7. The lyophilized formulation according to claim 3, comprising fosaprepitant or the pharmaceutically acceptable salt thereof, palonosetron or the pharmaceutically acceptable salt thereof, the human serum albumin, and the lyophilization excipient, wherein: each vial of the lyophilized formulation contains 220-270 mg of fosaprepitant dimeglumine, 0.25-0.31 mg of palonosetron hydrochloride, 30-300 mg of the human serum albumin, and 10-600 mg of the lyophilization excipient; preferably, each vial of the lyophilized formulation contains 220-270 mg of fosaprepitant dimeglumine, 0.25-0.31 mg of palonosetron hydrochloride, 60-200 mg of the human serum albumin, and 50-500 mg of the lyophilization excipient; further preferably, each vial of the lyophilized formulation contains 230-260 mg of fosaprepitant dimeglumine, 0.26-0.30 mg of palonosetron hydrochloride, 100-140.0 mg of the human serum albumin, and 100-400 mg of the lyophilization excipient.

8. The lyophilized formulation according to claim 1, wherein a proper amount of a pH adjuster is also required to be added during preparation, wherein the pH adjuster is an alkaline pH adjuster selected from one or more of sodium hydroxide, potassium hydroxide, sodium carbonate, sodium bicarbonate, disodium hydrogen phosphate, sodium dihydrogen phosphate, triethylamine, ethylenediamine, triethanolamine, Tris (tris(hydroxymethyl)aminomethane), arginine, lysine, histidine, glycine, or meglumine, and is preferably sodium hydroxide.

9. The lyophilized formulation according to claim 1, wherein the lyophilized formulation is a white, off-white, or light yellow lyophilized lump or powder.

10. The lyophilized formulation according to claim 1, wherein the lyophilized formulation is a clear solution after being reconstituted and diluted to be a drug solution for clinical infusion.

11. A method for preparing the lyophilized formulation according to claim 8, wherein the method comprises the following steps:
(1) dissolving fosaprepitant dimeglumine, the human serum albumin, and the lyophilization excipient in water for injection, adjusting the pH of the drug solution to a proper range by using a proper amount of sodium hydroxide, and supplementing with the water for injection to bring the volume to a total formulating amount, thus obtaining an intermediate drug solution;
(2) filtering and sterilizing the intermediate drug solution, filling, partially stoppering, and lyophilizing to obtain a product;
or, as an alternative embodiment, the method comprises the following steps:
(1) dissolving the lyophilization excipient in water for injection to obtain a first solution;
(2) adding the human serum albumin to the first solution and uniformly stirring to obtain a second solution;
(3) under continuous stirring, adding fosaprepitant dimeglumine to the second solution to gradually dissolve fosaprepitant dimeglumine, adjusting the pH of the drug solution to a proper range by using sodium hydroxide, and supplementing with the water for injection to bring the volume to a total formulating amount, thus obtaining an intermediate drug solution;
(4) filtering and sterilizing the intermediate drug solution, filling, partially stoppering, and lyophilizing to obtain a product;
or, as an alternative embodiment, the method comprises the following steps:
(1) dissolving the lyophilization excipient and the human serum albumin in water for injection to obtain a first solution;
(2) under continuous stirring, adding fosaprepitant dimeglumine to the first solution to gradually dissolve fosaprepitant dimeglumine, adjusting the pH of the drug solution to a proper range by using sodium hydroxide, and supplementing with the water for injection to bring the volume to a total formulating amount, thus obtaining an intermediate drug solution;
(3) filtering and sterilizing the intermediate drug solution, filling, partially stoppering, and lyophilizing to obtain a product.)

12. (canceled)

13. The method for preparing the lyophilized formulation according to claim 11, wherein the pH of the intermediate drug solution is adjusted to be in the range of 7.5 to 10.0 by the pH adjuster; preferably, the pH of the intermediate drug solution is adjusted to be in the range of 8.0 to 9.5 by the pH adjuster; further preferably, the pH of the intermediate drug solution is adjusted to be in the range of 8.5 to 9.0 by the pH adjuster.

14. A method for clinical use of a lyophilized formulation, wherein the lyophilized formulation is the lyophilized formulation according to claim 1, and the method of clinical use comprises the following steps:
(1) slowly injecting 5 mL of a solvent into the product along the vial wall with a syringe and slightly shaking the vial to assist dissolving;
(2) after lyophilized powder is completely dissolved, drawing a drug solution obtained by reconstitution with a syringe and injecting into an infusion bag or an infusion bottle containing 145 mL of a solvent, and slightly turning the infusion bag or the infusion bottle by hand 3 to 5 times to uniformly mix the drug solution, thus obtaining a drug solution for clinical infusion;
the solvent in the method for clinical use of a lyophilized formulation comprises one of sterile water for injection, 5% glucose injection, 0.9% sodium chloride injection, and 5% glucose and sodium chloride injection, and is preferably 0.9% sodium chloride injection.
